(19) United States
(12) Patent Application Publication
(10) Pub. No.: US 2005/0009054 A1
(43) Pub. Date: Jan. 13, 2005

(54) Compounds having a fused, bicyclic moiety for binding to the minor groove of dsDNA

(75) Inventors: Dennis P. Phillion, St. Charles, MO (US); James K. Bashkin, St. Louis, MO (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Pharmacia Corporation

(21) Appl. No.: 10/835,054

(22) Filed: Apr. 29, 2004

Related U.S. Application Data
(60) Provisional application No. 60/466,477, filed on Apr. 30, 2003.
(60) Provisional application No. 60/482,692, filed on Jun. 26, 2003.

(51) Int. Cl. C12Q 1/68; C07H 21/04

(52) U.S. Cl. 435/6; 536/23.1

(57) ABSTRACT

The present invention is directed to the means by which to alter the binding affinity and/or specificity of a compound with a sequence of DNA in the minor groove of a double-strand thereof. More particularly, the present invention is directed to a synthetic and/or non-naturally occurring compound (e.g., an analog of a polyamide oligomer or polymer) which contains at least one hydrogen bond donor moiety and at least one hydrogen bond acceptor moiety, wherein the latter moiety or “building block” has a fused, bicyclic structure which is heteroaromatic, said structure having a heteroatom therein which acts as a hydrogen bond acceptor to bind guanine in the minor groove of the dsDNA sequence, and which is incapable of forming a tautomer. In one particular embodiment of the synthetic and/or non-naturally occurring compound, the fused, bicyclic structure occupies an initial or first terminal position within the compound.

### REFERENCE TO RELATED APPLICATION

This application claims priority from U.S. Provisional Application Ser. No. 60/466,477 (filed on Apr. 30, 2003), and U.S. Provisional Patent Application Ser. No. 60/482,292 (filed on Jun. 26, 2003), the entire contents of which are incorporated herein by reference.

### BACKGROUND OF THE INVENTION

The present invention is generally directed to the means by which to alter the binding affinity and/or specificity of a compound with a sequence of DNA in the minor groove of a double-strand thereof. More particularly, the present invention is directed to a synthetic and/or non-naturally occurring compound (e.g., an analog of a polyamide oligomer or polymer) which contains at least one hydrogen bond donor moiety and at least one hydrogen bond acceptor moiety, wherein the latter moiety or “building block” has a fused, bicyclic structure which is heteroaromatic, said structure having a heteroatom therein which acts as a hydrogen bond acceptor to bind guanine in the minor groove of the dsDNA sequence, and which is incapable of forming a tautomer. In one particular embodiment of the synthetic and/or non-naturally occurring compound, the fused, bicyclic structure occupies an initial or first terminal position within the compound, as further described and illustrated herein.

An understanding of the synthesis, the analysis, and the manipulation of DNA has led to a significant increase in the number of opportunities for the diagnosis and treatment of various illnesses and conditions. For example, the specific interaction of proteins, such as transcription factors, with DNA is now understood to control the regulation of genes, and hence, the regulation of cellular processes as well. (See, e.g., Roeder, R. G., TIBBS. 9, 327-335 (1996).) Furthermore, a wide variety of human conditions ranging from cancer to viral infection are recognized to arise from malfunctions in the biochemical machinery that regulates gene expression. (See, e.g., R. Tjian, Sci. Am., 2, 54-61 (1995).) Therefore, researchers have focused on identifying specific sequences of DNA that, as a result of biochemical malfunction or otherwise, cause disease, defect, and discomfort when expressed. This research has led to a better understanding of particular genetic processes, as well as the ways to treat and deal with these processes when they run awry.

In recent years, researchers have learned that certain chemical compounds can be used to regulate the phenotypic effects of the genetic machinery. The expression of proteins, the end product of nucleic acid translation, can be controlled by the application of certain natural and synthetic compounds. The discovery and application of these chemicals have been to the benefit of both research and therapeutics. In research, these molecules can be used to modulate the activity of a particular gene in order to identify the function and cellular characteristics of that particular gene. In therapeutics, these molecules can be used to inhibit the proliferation of cells which may act as pathogens, where proliferation has an adverse effect on the host, or to combat diseases, including life threatening diseases, which result from misregulation in transcription.

It is well known that chemical compounds known as polyamides can be used to control gene expression due to their high affinity for DNA. Polyamides comprise polymers of amino acids covalently linked by amide bonds. Specific polyamides that target unique DNA sequences can be used to suppress or enhance the expression of particular genes, while not affecting the expression of others. More specifically, expression of a gene occurs when transcription compounds such as activators, transcription binding proteins, transcription factors, and the like bind to specific locations in the gene's promoter region known as transcription binding sites and either initiate or inhibit the process of DNA transcription. Administration of polyamides designed to bind to specific transcription binding sites in a gene's promoter region may therefore prevent the transcription regulators of a cell from binding to the transcription binding sites, thereby resulting in modulation of a gene expression.

It has become known that certain oligomers of nitrogen heterocycles can be used to bind to particular regions of double stranded DNA (“dsDNA”). Particularly, N-methyl imidazole (Im) and N-methylpyrrole (Py) have a specific affinity for particular bases. This specificity can be modified based upon the order in which these two compounds are connected via amide or amido (i.e., —NHC(O)—) linkages or groups. For example, it has been shown that there is specificity in that G/C is complemented by Im/Py, C/G is complemented by Py/Im, and A/T and T/A are redundantly complemented by Py/Py. In effect, N-methylimidazole tends to be associated with guanine, while N-methylpyrrole is associated with cytosine, adenine, and thymine. By providing for two chains of the heterocycles, as one or two molecules, a 2:1 complex with double-stranded DNA is formed, with the two chains of the oligomer antiparallel, where G/C pairs have Im/Py in juxtaposition, C/G pairs have Py/Im in juxtaposition, and T/A pairs have Py/Py in juxtaposition. The heterocycle oligomers are joined by amido (i.e., —NHC(O)—) groups, where the NH may participate in hydrogen bonding with nitrogen or oxygen unpaired electrons of nucleotide bases present in the floor of the DNA minor groove.

In those instances wherein two chains of heterocycles are present as one molecule, these chains may be so linked or synthesized to form “hairpin” compounds by incorporating, for example, γ-aminobutyric acid, to allow the single polyamide to form an antiparallel complex with DNA. Such a structure has been found to increase the binding affinity and selectivity of the polyamide to a target sequence of DNA.

More recently, it has been discovered that the inclusion of 3-hydroxy-N-methylpyrrole (Hp) can also act to increase selectivity in binding DNA base pairs; for example, when incorporated into a polyamide and paired opposite Py, Hp provides the means by which to discriminate A-T from T-A. (See, e.g., White S., et al., Nature 391 436-438 (1998).) Unexpectedly, the replacement of a single hydrogen atom on the pyrrole with a hydroxy group in an Hp/Py pair regulates the affinity and the specificity of a polyamide by an order of magnitude. Utilizing Hp together with Py and Im in polyamides to form four aromatic amino acid pairs (Im/Py, Py/Im, Hp/Py, and Py/Hp) provides a code to distinguish all four Watson-Crick base pairs in the minor groove of DNA.

Other compounds may also or alternatively be included in the polyamide, such as for example β-alanine (“β”). β-Alanine may be opposite either another β-alanine or a Py to selectively bind to an A/T or T/A base pair. (See, e.g., L. A. Dickenson et al., J. of Biological Chem., vol. 274, pp. 12765-12773 (1999).)

### SUMMARY OF THE INVENTION

Briefly, therefore, the present invention is directed, in one embodiment, to a synthetic and/or non-naturally occurring compound which binds a sequence of nucleotides with specificity in a minor groove of double-stranded DNA (“dsDNA”), said sequence containing at least one guanine nucleotide. In one embodiment, the compound comprises at least one hydrogen bond (“H-bond”) donor moiety and at least one H-bond acceptor moiety spaced apart to bind with specificity a sequence of nucleotides in a minor groove of dsDNA, wherein said H-bond acceptor moiety has a fused, bicyclic structure and is heteroaromatic, wherein said structure has a heteroatom therein which acts as a hydrogen bond acceptor to bind guanine in the minor groove of the dsDNA sequence, and wherein said structure cannot form a tautomer in which said heteroatom becomes a H-bond donor. In one particular embodiment, the fused, bicyclic structure occupies an initial or first terminal position within the compound. In this or other embodiments, the compound comprises two or more of such non-tautomerizing, fused, bicyclic structures, which may be the same or substantially the same, or alternatively are different.

The present invention is further directed, in one embodiment, to such a compound which is an analog of a polyamide oligomer or polymer, as further described herein. In these or other embodiments, the compound may have the structure: 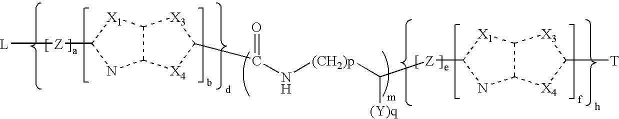
wherein:
L is independently selected from H, H₂N(HN)CNHCH₂, the terminal methylene group, CH₂, being attached to the remaining portion of the compound, and a non-tautomerizing, fused bicyclic structure: 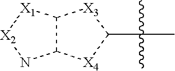
and further wherein each ring of each non-tautomerizing fused, bicyclic structure is unsaturated and has 5-members or 6-members, provided both rings are not 5-member rings;
X₁ and X₂ are independently selected from O, S, N, NR², CR³, CR⁴═CR⁴¹, CR⁴═N, N═CR⁴, N═N and CR⁴, provided that (i) when each one of X₁ or X₂ is independently selected from O, S or NR², the other is independently selected from CR³ or N, and (ii) when each one of X₁ or X₂ is independently selected from CR⁴═C R⁴, CR⁴═N, N═CR⁴ or N═N, the other is independently selected from CR⁴″, or N;
X₃ is independently selected from N, O, S, CR⁵, NR⁵, CR⁵═CR⁵′, CR⁵═N, N═CR⁵ and N═N, and X₄ is independently selected from O, S, N and CH, provided that (i) when each X₃ is independently selected from CR⁵ or N, X₄ is independently selected from O or S, and (ii) when each X₃ is independently selected from O, S, NR⁵, CR⁵═CR⁵′, CR⁵═N, N═CR⁵ or N═N, X₄ is independently selected from CH or N;
T is an amido-containing structure: 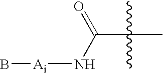
wherein A, when present, is independently selected from —CH₂CH₂C(O)— or —CH₂C(O)—, wherein the terminal methylene group is bound to nitrogen and the terminal carbonyl carbon is bound to B; and, B is independently selected from a diamine or triamine end-group;
Y, when present, is independently selected from H, NH₂, OH, SH, Br, Cl, F, OCH₃, CH₂OH, CH₂SH and CH₂NH₂;
Z is independently selected from (i)—C(O)NH-Q-, wherein Q is independently selected from substituted or unsubstituted C₁₋₆ alkyl, or (ii) one of structures (1), (2), (3) and (4): 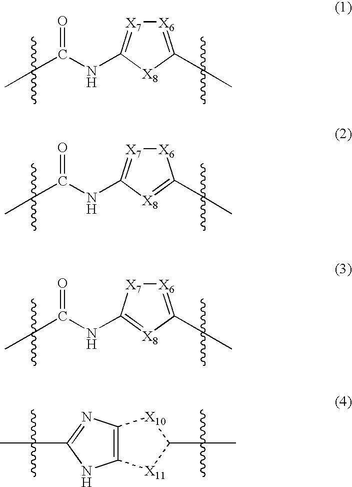
wherein
   for structure (1) X₆ is CR⁶, X₇ is independently selected from CR⁷ or N, and X⁸ is independently selected from O or S,
   for structure (2) X₆ is independently selected from NR⁶, O or S, X₇ is independently selected from CR⁷ or N, and X⁸ is independently selected from CH, C(OH), or N,
   for structure (3) X₆ is independently selected from CR⁶ or N, X₇ is independently selected from NR⁷, O or S, and X⁸ is independently selected from CH, C(OH), or N; and,
   for structure (4) each ring is unsaturated, X₁₀ is independently selected from CR¹⁰═CR¹⁰′, CR¹⁰═N, N═CR¹⁰ or N═N, and X₁₁ is independently selected from CH, C(OH), or N;
each substituent R², R³, R⁴, R⁴, R⁴¹¹, R⁵, R⁵′, R⁶, R⁷, R¹⁰ and R¹⁰′ is independently selected from H, hydroxy, N-acetyl, benzyl, substituted or unsubstituted C₁₋₆ alkyl, substituted or unsubstituted C₁₋₆ alkylamine, substituted or unsubstituted C₁₋₆ alkyldiamine, substituted or unsubstituted C₁₋₆ alkylcarboxylate, substituted or unsubstituted C₂₋₆ alkenyl, substituted or unsubstituted C₂₋₆ alkynyl and, when attached to a carbon atom, optionally halo, provided that (i) when X¹ or X² is NR², R² is other than H, and (ii) when X³ is NR⁵, R⁵ is other than H; and,
subscripts a, b, d, e, f, h, i, and p are each, independently, greater than or equal to 0, and subscripts m and q are 0 or 1, provided that (i) when L is not a non-tautomerizing, fused, bicyclic structure, b or f is at least about 1, (ii) when m is 0, q and p are also 0; (iii) the result of [(a+b)*d] is at least about 2; and, (vi) the result of [(e+f)*h] is the same or different from the result of [(a+b)*d] and is greater than or equal to 0, further provided that when the result of [(e+f) *h] is 0, m is 0.

The present invention is still further directed to compositions wherein a derivative of one of the above-described compounds is a moiety or component therein. For example, the present invention is further directed to: (i) a polyamide analog for binding a sequence of nucleotides with specificity in a minor groove of dsDNA, said polyamide analog comprising at least two derivatives of the above-described compounds, which may be the same or different, linked to form a tandem unit; (ii) a triplex comprising a dsDNA sequence to which is bound, in a minor groove thereof, a compound, or derivative thereof (as described above); and, (iii) a cell comprising such a triplex (e.g., a eukaryotic cell (e.g., mammalian), or a prokaryotic cell (e.g., a bacteria)).

The present invention is still further directed to processes wherein one of the above-described compounds is employed. For example, the present invention is further directed to a process for forming a triplex between a sequence of nucleotides in a minor groove of a dsDNA and a compound (or polyamide analog thereof) of the present invention which is designed to bind said sequence with specificity. The process comprises (i) identifying said sequence; (ii) contacting said sequence with a compound as described above; and, (iii) forming a triplex of the compound and the sequence of nucleotides of the dsDNA, wherein said compound forms H-bonds with nucleotide base pairs in the minor groove of the dsDNA, and further wherein a fused bicyclic moiety of the compound forms a H-bond with a G nucleotide in the sequence by means of the heteroatom therein which acts as a H-bond acceptor.

The present invention is still further directed to a process of detecting a dsDNA composition in a sample. The process comprises (i) contacting, under triplex-forming conditions, a sample of dsDNA and a compound (or polyamide analog thereof) as described above, said compound further comprising a moiety for detecting triplex formation between said dsDNA and said compound; and, (ii) detecting the presence of dsDNA in said sample as a triplex with said compound by means of said detectable moiety. In a preferred embodiment, the detectable moiety is an enzyme, a solid surface, a hapten which binds to a receptor, a radioactive isotope, or some other moiety that is detectable by means of fluorescence or chemiluminescence.

The present invention is still further directed to a process of separating a specific dsDNA from a mixture of dsDNA. The process comprises (i) contacting, under triplex-forming conditions, a mixture of dsDNA and a compound (or polyamide analog thereof) as described above, said compound further comprising a moiety for separating a triplex formed between said specific dsDNA and said compound; and, (ii) separating a triplex formed between said specific dsDNA in said mixture with said compound by means of said separation moiety (such as, for example, a hapten).

The present invention is still further directed to a process for regulating proliferation of cells in a mammalian host. The method comprises administering a proliferation-regulating amount of a compound (or polyamide analog thereof) as described above, wherein (i) a dsDNA, which is all or part of a target gene essential for proliferation of said cells, comprises a sequence of nucleotides which said compound binds with specificity thereto, and (ii) said compound so binds to said site by forming H-bonds with nucleotide base pairs in the minor groove of said dsDNA, a fused bicyclic moiety of said compound forming a H-bond with a G nucleotide in said dsDNA sequence by means of the heteroatom therein which acts as a H-bond acceptor for said G nucleotide, thereby regulating transcription of said gene and controlling the proliferation of said cells.

The present invention is still further directed to a composition which includes one of the compounds (or polyamide analog thereof) described above, such as a composition for regulating transcription. For example, such a composition may comprise a pharmaceutically acceptable excipient and a transcription-regulating amount of a compound suitable for binding a sequence of nucleotides (which comprises 1 or more guanine nucleotides) in the minor groove of dsDNA with specificity, as described herein. The present invention is still further directed to a method of treating a subject having a condition associated with the expression or over-expression of an oncogene comprising administering such a composition.

The present invention is still further directed to a process for regulating transcription of a gene in a cell in an organism. The method comprises administering to said organism or cell a transcription-regulating amount of at least one compound (or polyamide analog thereof) as described above, wherein (i) a dsDNA, which is all or part of said gene, comprises a sequence of nucleotides which said compound binds with specificity thereto, and (ii) said compound so binds said sequence by forming H-bonds with nucleotide base pairs in the minor groove of said dsDNA, a fused bicyclic moiety of said compound forming a H-bond with a G nucleotide in said dsDNA sequence by means of the heteroatom therein which acts as a H-bond acceptor for said G nucleotide, thereby regulating transcription of said gene in said organism or cell.

The present invention is still further directed to a process for regulating replication of a pathogen, the process comprising administering a transcription-regulating amount of a compound (or polyamide analog thereof) as described herein (e.g., an analog of a polyamide oligomer or polymer) which is suitable for binding a sequence of nucleotides in a minor groove of a dsDNA essential for replication of said pathogen.

The present invention is still further directed to a process for modulating the expression of a cellular or viral gene. The process comprises (i) identifying a nucleotide sequence in a dsDNA adjacent to a binding site of at least about one transcription factor protein in a minor groove of said dsDNA, said sequence comprising at least one guanine nucleotide; (ii) choosing a synthetic and/or non-naturally occurring compound, or polyamide analog) as described above; and, (iii) contacting said target sequence with a transcription modulating amount of said compound (or polyamide analog).

The present invention is still further directed to a process for preparing a compound as described herein, on a solid support. The process comprises (a) preparing a support for attachment of said compound; (b) reacting an amino acid with a reagent to provide an amino acid containing an amino group which is protected and a carboxyl group reactive with an amino functionality; (c) sequentially deprotecting the amino acid and adding the protected and reactive amino acids to the solid support beginning with the carboxy terminal amino acid, thereby forming the desired compound; (d) cleaving the compound from the resin; and, (e) purifying the compound, wherein at least one of said protected and sequentially deprotected amino acids comprises a fused, bicyclic structure having a 5- or 6-member heteroaromatic ring, wherein said structure has a heteroatom therein which acts as a hydrogen bond acceptor to bind guanine in the minor groove of dsDNA, and further wherein said structure cannot form a tautomer in which said heteroatom becomes a H-bond donor.

It is to be noted that in one or more of the above embodiments, or described elsewhere herein, the compound may comprise one or more fused, bicyclic structures which may be the same or substantially the same, or different.

### BRIEF DESCRIPTION OF THE DRAWINGS

FIG. 1 illustrates two analogs of I—P—I—P—β—Dp that incorporate a fused, bicyclic structure of the present invention (wherein, as used herein, I═Im═N-methylimidazole, P═Py═N-methylpyrrole, and β═β-alanine, and further wherein the large, open spheres represent the nucleotide atoms which H-bond with the polyamide and the large, lined-through spheres represent the polyamide nitrogen atoms that H-bond with DNA).

FIGS. 2 and 3 are graphs, as further discussed in Example 7, which illustrates polyamide (or polyamide analog) inhibition (IC₅₀ values) of in vitro transcription-translation assays of a number of compounds prepared in Example 6 (wherein, for FIG. 2: IP₂IGP₄BDa, IC₅₀=1.18 μM (average), and for FIG. 3: IP₂IGP₄BDa, IC₅₀=2.60 μM (average), BiPBBiGP₄BDa, IC₅₀=50.22 μM (average), BiP₂BiGP₄BDa, IC₅₀=19.83 μM (average), and IP₂BiGP₄BDa, IC₅₀=124 μM (extrapolated)).

### DETAILED DESCRIPTION OF THE PREFERRED EMBODIMENTS

In accordance with the present invention, a new compound having binding affinity and/or selectivity, for binding a sequence of nucleotides in the minor groove of dsDNA, has been discovered. The compound of the present invention comprises at least one H-bond donor moiety and at least one H-bond acceptor moiety, wherein the latter has a heteroaromatic fused, bicyclic structure (i.e., wherein one of the rings thereof is heteroaromatic and the other is aromatic or heteroaromatic), said structure having a heteroatom therein which acts as a hydrogen bond acceptor to bind guanine in the minor groove of dsDNA, and further wherein said structure cannot form a tautomer in which said heteroatom becomes a H-bond donor.

In one embodiment, associated with, or bound directly or indirectly to, this fused, bicyclic structure are optionally other cyclic or heterocyclic compounds, which may or may not serve has H-bond donors or acceptors. Additionally, the compounds of the present invention may comprise linking moieties (e.g., H-bond donors, such as amido (i.e., —C(O)NH—) or amido-containing linking moieties). Accordingly, in one embodiment the compound of the present invention may comprise a series of at least about 2, 4, 6, 8, 10 or more cyclic moieties (e.g., heterocyclic, including heteroaromatic, moieties and fused, bicyclic structures as described herein), ranging from example from about 2 to about 10, or about 4 to about 8, which are bound with one or more linking moieties, in order to form a complementary pairing with target nucleotides of the dsDNA.

In this regard it is to be noted that, in some instances, the compounds of the present invention may be described as analogs of synthetic and/or non-naturally occurring polyamide oligomers or polymers, the binding affinity and/or selectivity potentially being improved, relative to conventional polyamides, by the inclusion of one or more moieties having said fused, bicyclic structure which serves as a H-bond acceptor. For example, the compounds of the present invention may alternatively be described as oligomers in those instances wherein they comprise at least about 2, 4, 6, 8, 10 or more H-bond donor and/or H-bond acceptor moieties, while the present compounds may alternatively be described as polymers when two or more of said oligomers are linked (e.g., multiple hairpin oligomers may be linked to form a polyamide, as described and/or illustrated elsewhere herein).

It is to be further noted that, as used herein, an “analog” of a polyamide oligomer or polymer generally refers to a polyamide oligomer or polymer, respectively, wherein one or more amido or amido-containing moieties, which are otherwise present to link the units (e.g., repeat units) thereof, are absent, typically being replaced by a bond directly linking one unit of the oligomer or polymer to the next. For example, in one embodiment of the compound of the present invention the fused, bicyclic structure is directly bound to another fused, bicyclic structure or a heterocyclic moiety (e.g., a pyrrole or imidazole ring). Accordingly, in the polyamide analogs of the present invention, the addition of each fused, bicyclic structure enables the elimination of a H-bond donor (e.g., an amido linker or amido-containing moiety). Thus, the present invention is directed to analogs of polyamides which are capable of altered, and preferably enhanced, interactions in the minor groove of dsDNA (as compared to conventional polyamides).

It is to be still further noted that “non-naturally occurring,” as used herein, is intended to refer to a compound which contains one or more nucleotide binding moieties (e.g., H-bond donor or acceptor moieties) that may not be found in nature within the same molecule. Additionally, “synthetic,” as used herein, is intended to refer to a compound which has been prepared using organic synthesis techniques (such as those further described and/or illustrated herein).

It is to be still further noted that “complementary,” as well as variations thereof, is intended to refer to a preferential juxtaposition of heterocycles and fused, bicyclic structures of the compound of the present invention with the nucleotides of the dsDNA.

It is to be still further noted that, although the compound of the present invention is generally referred to herein as a “minor groove” binder, it may not in all instances or embodiments exhibit binding interactions exclusively with the minor groove. For example, the compound may also exhibit binding interactions with other parts of the dsDNA (e.g., with backbone phosphate groups).

It is to be still further noted that “physiological conditions,” or variations thereof, generally refer to conditions which are common in physiological applications or settings. For example, in one embodiment, this term refers to conditions which are not sufficiently acidic to result in the protonation of the H-bond acceptor heteroatom in the fused, bicyclic structure (e.g., conditions wherein the pH is not less than about 7, about 6, about 5, or even about 4).

It is to be still further noted that a fused, bicyclic structure in the compound of the present invention which serves as a H-bond acceptor lacks the ability to form a tautomeric structure, wherein the heteroatom that is present therein to bind guanine participates; that is, this fused bicyclic structure cannot tautomerize, such that the heteroatom which is present therein to bind guanine become substituted with a hydrogen atom. Stated another way, this fused, bicyclic structure cannot become a H-bond donor.

It is to be still further noted that “specificity,” or variations thereof, generally refers to the preferential binding of the compound of the present invention to a given or “target” sequence of nucleotides in the dsDNA, as opposed to another sequence within the same dsDNA; stated another way, this refers to the ability of the compound of the present invention to more discriminately bind (i) sequences which contain a guanine nucleotide as compared to sequences that do not, and/or (ii) sequences which both contain guanine nucleotides, but in different numbers and/or locations within the sequences.

The compound of the present invention is believed suitable, for example, for use in compositions capable of being transported across cellular membranes to the nucleus, binding to DNA (e.g., chromosomal DNA), and fulfilling a variety of intracellular functions, including regulating (e.g., inhibiting) transcription. The compound, and/or compositions in which they are present, may be modified to be used in diagnostics, particularly by providing for detectable and/or isolatable labels, or may be used in research or therapeutics, to regulate (e.g., inhibit) transcription of, for example, target genes. These compounds and/or compositions may be otherwise modified to enhance properties for specific applications, such as transport across cell walls, association with specific cell types, cleaving of nucleic acids at specific sites, change chemical and physical characteristics, and the like.

I. Binding/Selectivity and H-bond “Slippage”

It is now well-recognized that heterocyclic amino carboxylic acids may be used to synthesize polyamides that bind to the minor groove of dsDNA. The N-methylpyrrole unit binds with adenine, thymine and cytosine, while the N-methylimidazole unit is specific for guanine. Without being held to any particular theory, it is believed that this specificity is achieved through two contributing factors. First, a positive interaction occurs when the G amino group located in the dsDNA minor groove H-bonds with the basic imidazole nitrogen facing the floor of the minor groove. Second, a negative interaction occurs when the N-methylimidazole is replaced with N-methylpyrrole, because of a steric repulsion between the G amino group located in the dsDNA minor groove and the pyrrole C—H facing the floor of the minor groove.

The binding of a polyamide with repeating pyrrole units to an A/T region of DNA occurs through H-bonding of regularly-spaced secondary amide hydrogens of the polyamide with specific A and T heteroatoms. The spacing between the secondary amide hydrogens is close or similar to the separation distance between the parallel planes of adjacent base pairs in B-DNA. In principle, therefore, the interaction of G with an imidazole unit may simultaneously occur through two separate interactions: (i) H-bonding of a G amino group located in the DNA minor groove with the basic imidazole nitrogen facing the floor of the minor groove; and, (ii) H-bonding of the imidazole amide N—H (at the 4-position) with a purine nitrogen of G. However, it is believed that both of these interactions may not simultaneously occur in the plane defined by a G/C base pair, and thus one interaction is expected to dominate. If the most important interaction is H-bonding of a G amino group located in the DNA minor groove with the basic imidazole nitrogen facing the floor of the minor groove, then the second interaction may play a minor role in overall binding affinity.

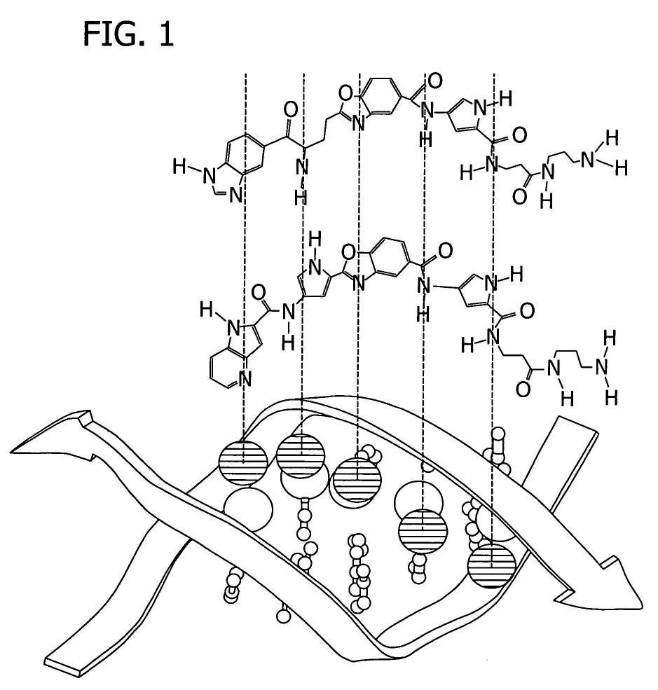

Without being held to a particular theory and referring now to FIG. 1, it is believed that the close register of interactions between the polyamide and the dsDNA target sequence is distorted when an imidazole is incorporated into the polyamide to recognize G, because the distance between an amide N—H and an imidazole N is much less than the distance between adjacent base pairs in B-DNA. The present invention therefore provides a new approach for G-specific recognition, wherein the H-bonding and the H-donating functionalities occur at substantially regular intervals along the length of a polyamide molecule. As a result, essentially all of the H-bond donating and accepting interactions of the polyamide may be in register with the spacing of the DNA base pairs. For example, in at least one embodiment of the present invention, wherein the compound comprises, in some combination, the fused, bicyclic structures as described herein as the H-bond acceptor moieties and pyrrole/amido linkers as the H-bond donor moieties, the number of bonds separating (i) H-bond donor moieties from each other, (ii) H-bond acceptor moieties from each other, and/or (iii) a H-bond donor moiety from a H-bond acceptor moiety, are about the same; for example, in one embodiment substantially all of these moieties (e.g., all moieties excluding those attached to the tail of the compound) are separated by at least about 2 bonds (e.g., about 3 bonds, about 4 bonds, or about 5 bonds). In contrast, the number of bonds separating H-bond donor and H-bond acceptor moieties in conventional compounds (i.e., polyamides) which comprise imidazole and pyrrole/amido moieties is different; that is, the number of bonds separating these moieties in conventional compounds is not the same.

Accordingly, it is believed that use of the fused, bicyclic structure described herein as a compound “cap” (i.e., placed in a first or initial terminal position within the compound, as further described herein), as well as in one or more internal (i.e., non-terminal positions) within the compound, may act to improve the overall registry of the compound in the minor groove of the dsDNA, thus acting to improve binding affinity. Furthermore, it is believed that such a compound may have improved selectivity, given that a moiety within, for example, a polyamide that may act as either a H-bond donor or H-bond acceptor has been removed and replaced by the fused, bicyclic structure, which may only act as a H-bond acceptor. As a result, the opportunity to bind an A, C or T nucleotide has been removed.

Additionally, it is believed that use of the fused, bicyclic structure described herein, to replace, or enable the removal of, an amido group may act to alter the uptake and/or movement of the overall compound within a cell. More specifically, the compounds of the present invention may have improved uptake and/or movement, as compared to a standard polyamide, given that amides tend to be easily moved or “pumped out” of a cell.

Finally, it is to be noted that in one embodiment the compounds of the present invention enable “slippage” to occur between the compound and the dsDNA; that is, in one embodiment the fused, bicyclic structure enables a shift or slip in the interactions between a H-bond donor in the dsDNA and the fused, bicyclic H-bond acceptor structure to occur. Without being held to a particular theory, it is generally believed that when the fused, bicyclic structure comprises two heterocyclic rings, wherein the heteroatoms therein are properly oriented and spaced apart, a shift in H-bonding interaction may occur as the H-bond donor of the dsDNA and the H-bond acceptor heteroatom in the first ring of the fused, bicyclic structure becomes progressively more out of register. At some point, this shift occurs, resulting in a new H-bond interaction between the H-bond donor of the dsDNA and the H-bond acceptor heteroatom in the second ring of the fused, bicyclic structure. In this way, the present invention enables longer compounds (e.g., polyamide analogs) to be utilized, while registry with the dsDNA is maintained. An exemplary embodiment of such a fused, bicyclic structure is: 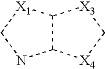
wherein X¹, X³ and X⁴ (which may be the same or different) are as further described herein, and provided: (i) X⁴ is a heteroatom as described herein (i.e., a H-bond acceptor heteroatom); and, (ii) each ring of the fused, bicyclic structure is unsaturated and has 5-members or 6-members (with the exception that both rings do not have 5-members).
II. The Compound

A. Non-Tautomerizing, Fused, Bicyclic H-bond Acceptor

Generally speaking, the heterocyclic (e.g., heteroaromatic) portions or moieties of the compound of the present invention (e.g., analogs of a polyamide oligomers and/or polymers), as well as the heteroaromatic portion of the fused, bicyclic structure therein (or more specifically the heteroatom-containing ring of the overall heteroaromatic fused, bicyclic structure), may have from about 1 to about 3 (e.g., about 1 or about 2) heteroatoms therein, which are typically selected from nitrogen, oxygen, sulphur or a combination thereof. In those instances wherein only one of the rings of the fused, bicyclic structure contains a heteroatom, this is preferably the first ring of the structure (i.e., the ring within a given fused, bicyclic structure which is sequentially farthest from the tail end of the compound), as opposed to the second ring (i.e., the ring within a given fused, bicyclic structure which is sequentially closest to the tail end of the compound).

In one particular embodiment, one or more of the heteroatoms in the heterocyclic portion of a fused, bicyclic structure is nitrogen, which may or may not be substituted. However, without being held to a particular theory, it is to be noted that substitution of the heteroatom is generally believed to be, at least in part, dependent upon whether the heteroatom (e.g., nitrogen) is directed toward or away from the floor of the minor groove of the dsDNA. This is because greater latitude in the nature of the substitution is generally believed to be permitted when the heteroatom is directed away from the floor of the minor groove, given that steric repulsion is less problematic.

In one embodiment of the present invention, a fused, bicyclic structure comprises a combination of fused 5-member and/or 6-member rings, and in particular 5-member heteroaromatic and/or 6-member aromatic or heteroaromatic rings. For example, the structure may comprise a 5-member and a 6-member ring (e.g., a 5/6 or a 6/5 ring system, wherein the first number indicates the size of the first ring and the second number indicates the size of the second ring), or alternatively two 6-member rings (e.g., a 6/6 ring system), one or both of the rings being heterocyclic (e.g., heteroaromatic).

In this regard it is to be noted however that, in such embodiments, the fused, bicyclic structure is typically other than two 5-member rings (e.g., a 5/5 ring system). Without being held to a particular theory, it is generally believed that a 5/5 fused, bicyclic ring structure may not enable a spacing and/or a conformation which is sufficiently suitable for purposes of enabling maximum binding affinity to the minor groove of dsDNA.

It is to be still further noted that the rings of each fused, bicyclic structure are unsaturated (i.e., aromatic or heteroaromatic). Without being held to a particular theory, it is generally believed that aromaticity or heteroaromaticity aids in maximizing binding affinity of the compound, because the fused, bicyclic structure is essentially planar and therefore is better suited for fitting within the minor groove of the dsDNA, the planar nature of the moiety aiding for example in reducing any steric hindrance that might otherwise be present.

The fused, bicyclic structure which serves as a H-bond acceptor may occupy an initial or first terminal position within the compound, the structure thus effectively acting as a “cap.” Alternatively, or additionally, one or more of these fused, bicyclic structures may occupy an internal (i.e., non-terminal) position within the compound. In those instances wherein multiple fused, bicyclic structures are present (e.g., two or more occupying non-terminal positions within the compound, or one acting as a cap and one or more occupying a non-terminal position), such structures may be the same, substantially the same (e.g., in those instances wherein the fused, bicyclic structures occupy both a terminal and a non-terminal position, the two differing only at one point of attachment of either the first or second ring therein), or different.

Additionally, in some embodiments such a fused, bicyclic structure may be bound on one or both sides to a H-bond donor (e.g., an amido linker or a linking moiety comprising an amido group), for example such as when the structure occupies a non-terminal position within the compound, while in other embodiments the fused, bicyclic structure is not so bound. For example, in some embodiments one or both rings of the fused, bicyclic structure may be bound directly (i.e., no intervening moiety is present) to another heterocyclic moiety or another fused, bicyclic structure.

In view of the foregoing, it is to be noted that the fused, bicyclic structure which serves as a H-bond acceptor in the compound of the present invention may be characterized, in one embodiment, as: 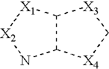
wherein:
X₁ and X₂ are independently selected from O, S, N, NR², CR³, CR⁴═CR⁴′, CR⁴═N, N═CR⁴, N═N and CR⁴″, provided that (i) when each one of X₁ or X₂ is independently selected from O, S or NR², the other is independently selected from CR³ or N, and (ii) when each one of X₁ or X₂ is independently selected from CR⁴═CR⁴′, CR⁴═N, N═CR⁴ or N═N, the other is independently selected from CR⁴″ or N;
X₃ is independently selected from N, O, S, CR⁵, NR⁵, CR⁵═CR⁵′, CR⁵═N, N═CR⁵ and N═N, and X₄ is independently selected from O, S, N and CH, provided that (i) when each X₃ is independently selected from CR⁵ or N, X₄ is independently selected from O or S, and (ii) when each X₃ is independently selected from O, S, NR⁵, CR⁵═CR⁵′, CR⁵═N, N═CR⁵ or N═N, X₄ is independently selected from CH or N; and,
each R substituent (i.e., R², R³, R⁴, R⁴′, R⁴¹′, R⁵, R⁵′) generally represents a hydrogen or some other substituent, as defined herein, which does not detrimentally hinder binding of the oligomer to the dsDNA or, alternatively, acts to enhance such binding, provided that the structure cannot form a tautomer in which the heteroatom for binding guanine becomes a H-bond donor (e.g., when X¹ or X² is NR², R² is other than H, and when X³ is NR⁵, R⁵ is other than H).
   In this regard it is to be noted that the dotted lines in the above structure indicate that the rings are unsaturated (aromatic or, in the case of a heteroatom-containing ring, heteroaromatic). As detailed elsewhere herein, and in view of the exceptions noted above and elsewhere herein, acceptable substituents (e.g., R) may include, for example, those independently selected from H, hydroxy, N-acetyl, benzyl, substituted or unsubstituted C₁₋₆ alkyl, substituted or unsubstituted C₁₋₆ alkylamine, substituted or unsubstituted C₁₋₆ alkyldiamine, substituted or unsubstituted C₁₋₆ alkylcarboxylate, substituted or unsubstituted C₂₋₆ alkenyl, substituted or unsubstituted C₂₋₆ alkynyl, and the like, and when attached to a carbon optionally halo (e.g., chloro).

In this regard it is to be noted that, as further illustrated herein below, in those instances or embodiments wherein the first ring of the fused, bicyclic structure is heteroaromatic and the structure occupies an initial terminal (i.e., “cap”) position, X₂ may be, for example, C—H when X₁ is NR² (e.g., N—CH₃). Conversely, in those instances or embodiments wherein the first ring of the fused, bicyclic structure is heteroaromatic and the structure occupies an internal (i.e., non-terminal) position within the oligomer and X₁ is NR² (e.g., N—CH₃), X₂ may be C═, the carbon atom represented by X₂ forming a double-bond with the adjacent, non-X₁, nitrogen atom in the ring.

Accordingly, when the fused, bicyclic structure serves as a cap (i.e., occupies a first terminal position within the compound), typically the carbon present between X₃ and X₄ is the point of attachment of the second ring to the remaining portion of the compound. In such instances, the structure may be represented, for example, as: 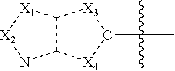
wherein the bond extending from the noted sp² hybridized carbon serves to connect the structure to the remaining portion of the compound (the curved line extending through the bond from the carbon atom shown indicating here, and in all other structures provided herein, that the remaining portion of the structure, to which this atom (a carbon atom here) is attached, is not shown). It is to be further noted that when the fused, bicyclic structure alternatively, or additionally (when more than one is present), occupies an internal (i.e., a non-terminal) position within the compound, X₂ is typically a sp² hybridized carbon atom which serves as a point of attachment to the compound for the first ring of the structure, while the Sp² hybridized carbon atom between X₃ and X₄ serves as the point of attachment for the second ring thereof (as described above). In such instances, the structure may be represented, for example, as: 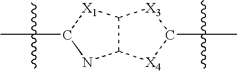
wherein the bond extending from each of the sp² hybridized carbon atoms serve to connect the structure to the remaining portion of the oligomer or polymer. However, in this regard it is to be still further noted that the point, or points, of attachment of the fused, bicyclic structure may be other than herein described without departing from the scope of the present invention.

It is to be further noted that, in those embodiments wherein a non-tautomerizing, fused, bicyclic structure occupies a terminal position as well as one or more non-terminal positions, (i) the terminal structure and one or more of the non-terminal structures may be substantially the same (i.e., differing essentially only with respect to one point of attachment), or different, and/or (ii) the non-terminal structures (when more than one is present) may be the same or different.

In view of the foregoing, and as previously noted, in some preferred embodiments both rings of the fused, bicyclic structure may be aromatic or heteroaromatic. Further, in such embodiments, for example: (i) X₁ is N—CH₃ and X₂ is CH or C═(wherein, for example, the fused, bicyclic structure is a cap or occupies a non-terminal position, respectively, within the compound), and/or X₃ is CR⁵═CR⁵′ (wherein R⁵ and R⁵′ are typically H) and X₄ is CH; (ii) X₁ is CR⁴═CR⁴′, (wherein R⁴ and R⁴′ are typically H) and X₂ is CH, and/or X₃ is N—CH₃ and X₄ is CH; or, (iii) X₁ is S and X₂ is CH or C═(wherein, for example, the fused, bicyclic structure is a cap or occupies a non-terminal position, respectively, within the compound), and/or X₃ is CR⁵═CR⁵″ (wherein R⁵ and R⁵′ are typically H) and X₄ is CH.

B. Non-fused, Non-bicyclic Heterocycles

In some embodiments, the compound of the present invention typically comprises at least about 2, 4, 6, 8, 10 or more heterocyclic moieties (e.g., heteroaromatic moieties), at least one of these moieties being part of a larger fused, bicyclic structure as described herein. Additionally, in some embodiments the compound may comprise less than about 50, about 40, about 30, about 20, or even about 10 heterocyclic and/or heteroaromatic moieties. Accordingly, in some embodiments the compound may comprise about 2 to about 50, about 4 to about 40, about 6 to about 30 or even about 8 to about 20 heterocyclic and/or heteroaromatic moieties, while in other embodiments the compound may comprise about 2 to about 10 or about 4 to about 8 heterocyclic and/or heteroaromatic moieties, with at least about 1, 2, 4, 6, 8, 10 or more of the heterocycles being part of a fused, bicyclic structure.

In some embodiments, one or more (e.g., about 2, 4, 6, 8, 10 or more) of the heterocycles are non-fused, non-bicyclic rings having about 5- or 6-members. Additionally, they may have from about 1 to about 3 (e.g., about 1 or about 2) heteroatoms therein. In those instances wherein more than one heteroatom is present, these heteroatoms may be adjacent or bound to each other, or alternatively spaced apart by at least about 1 intervening carbon atom, and in some instances about 2 intervening carbon atoms. These non-fused, non-bicyclic heterocycles may be completely unsaturated (e.g., heteroaromatic). Further, in some embodiments, the heterocycles may be linked, for example, at the 2-position and the 4- or 5- position (e.g., in the case of 5-member ring, the 2- and 4-position), to the remaining portion of the compound (e.g., to another heterocycle, including a fused, bicyclic structure, or a linker, as further described herein).

Among the non-fused, non-bicyclic heterocyclic or heteroaromatic moieties that may be present in some embodiments of the compound are, for example, substituted or unsubstituted pyrrole, substituted or unsubstituted furan, substituted or unsubstituted thiophene, substituted or unsubstituted pyrazole, substituted or unsubstituted oxazole, substituted or unsubstituted thiazole, substituted or unsubstituted isoxazole, substituted or unsubstituted isothiazole, and/or a combination thereof, while in other embodiments these moieties may be substituted or unsubstituted imidazole, substituted or unsubstituted triazole, substituted or unsubstituted oxadiazole, substituted or unsubstituted thiadiazole, substituted or unsubstituted cyclopentadiene, substituted or unsubstituted pyridine, substituted or unsubstituted pyrimidine, substituted or unsubstituted triazine and the like, and/or a combination thereof. In one preferred embodiment, the non-fused, non-bicyclic heterocyclic or heteroaromatic moiety or moieties are substituted or unsubstituted pyrrole and/or substituted or unsubstituted imidazole.

In one particular embodiment, the heterocyclic or heteroaromatic moiety contains one or more heteroatoms (e.g., nitrogen), which may or may not be substituted. However, as previously noted and without being held to any particular theory, substitution of the heteroatom is generally believed to be, at least in part, dependent upon whether the heteroatom (e.g., nitrogen) is directed toward or away from the floor or surface of the minor groove of the dsDNA, greater latitude in the nature of the substitution is generally believed to be permitted when the heteroatom (e.g., nitrogen) is directed away from the floor of the minor groove.

C. Substituents

As for the substituents which may optionally be present in the compound generally, and on one or more of the non-fused, non-bicyclic rings in particular, it is to be noted that such substituents are, in some embodiments, present at positions on a given heterocycle which are directed away from the floor of the minor groove of the dsDNA. For example, a hydrogen atom may be replaced with a substituent of interest, where the substituent will not result in increased steric interference with the floor or wall of the minor groove or otherwise create repulsion therewith. When substituted, the substituents may vary widely, being for example (i) a heteroatom, (ii) a hydrocarbyl, of typically from about 1 to about 30, and more usually about 1 to about 20, about 1 to about 10, or even about 1 to about 5 carbon atoms, including for example aliphatic, cyclic, aromatic, and/or combinations thereof, including both aliphatic saturated and unsaturated, (iii) a hetero-substituted hydrocarbyl, having for example from about 1 to about 10, about 1 to about 8, or about 1 to about 5 heteroatoms, including aliphatic, cyclic, aromatic and heterocyclic, and combinations thereof, where the heteroatoms are exemplified by halogen, nitrogen, oxygen, sulfur, phosphorous, and the like.

In this regard it is to be noted that, in those instances wherein an unsaturated substituent is present, in some embodiments typically not more than about 20%, about 15%, about 10% or even about 5% of the carbon atoms participate in aliphatic unsaturation.

In those instances wherein one or more atoms (e.g., a heteroatom such as nitrogen) in one of the moieties of the compound (e.g., a heterocycle or fused, bicyclic structure of the compound) is substituted, exemplary substituents include hydroxy, acetyl, substituted or unsubstituted aryl (e.g., phenyl or benzyl), substituted or unsubstituted alkyl (e.g., C₁₋₆ alkyl, such as methyl, ethyl, propyl, etc.), substituted or unsubstituted alkylamine (e.g., C₁₋₆ alkylamine), substituted or unsubstituted alkyldiamine (e.g., C₁₋₆ alkyldiamine), substituted or unsubstituted alkylcarboxylate (e.g., C₁₋₆ alkylcarboxylate), substituted or unsubstituted alkenyl (e.g., C₂₋₆ alkenyl), substituted or unsubstituted alkynyl (e.g., C₂₋₆ alkynyl), and the like, and when attached to a carbon atom the substituent may additionally be selected from such a group which also includes halo (e.g., chloro, bromo, fluoro, iodo).

For some embodiments, individual substituents will be less than about 750 Dal, less than about 500 Dal, less than about 250 Dal, or even less than about 100 Dal, in size. Additionally, the total carbon atoms for the substituent(s) will, in some embodiments, not be greater than about 100, about 75, about 50, or even about 25, with not more than about 20 heteroatoms, about 10 heteroatoms, or even about 5 heteroatoms present therein.

As previously noted, the substituents present on the compound (i.e., substituents present on a cyclic or heterocyclic moiety, a fused, bicyclic moiety, and/or a linking moiety thereof) are typically selected so as to avoid significant interference with compound binding in the minor groove. Additionally, substituent selection (e.g., by employing a single stereoisomer) may be utilized in order to impart certain desired properties to the subject compound, such as water solubility, lipophilicity, non-covalent binding to a receptor, radioactivity, fluorescence, and the like (as further described herein, see for example the discussion below about optional tail or end groups).

D. Linkers/Non-cyclic Oligomer Moieties

In at least one embodiment, the compound of the present invention comprises one or more fused, bicyclic structures, and optionally one or more cyclic or heterocyclic (e.g., heteroaromatic) structures, to which is bound or interposed there between a linking moiety or group capable of acting as a H-bond donor for purposes of binding with, for example, an unshared pair of electrons associated with for example an A or a T nucleotide. Accordingly, in some embodiments the compound additionally comprises an amido group or an amido-containing group (or, more generally, a group or moiety which may act as a H-bond donor, including for example groups or moieties having a —NH— therein, such as —CH₂NH—, —C(S)NH— and/or a benzimidazole). Alternatively, or additionally, the compound may comprise one or more other groups, such as methyleneamino, thiocarbonylamino, and imidinyl (or amidines).

In addition to the cyclic or heterocyclic compounds, as well as the linkers noted above, in one or more embodiments of the present invention the compound may optionally comprise an aliphatic amino acid (e.g., an Ω-amino aliphatic amino acid) in order, for example: (i) to enable a hairpin turn, or alternatively a γ-turn (using, for example, γ- or 2,4-aminobutyric acid) to provide complementation between two sequences of heterocycles, and/or to introduce or provide a chiral center in the compound (i.e., the turn has a chiral center therein, the center being introduced by means of, for example, the use of R-2,4-aminobutyric acid as the aliphatic amino acid); (ii) to form a cyclic compound (wherein the compound is joined at both ends); or, (iii) to provide for a shift in spacing of the organic cyclic compounds in relation to the sequence of nucleotides of the dsDNA to which the compound is to specifically or preferentially bind. In some embodiments, the aliphatic amino acids may have a chain as a core structure of about 2 to about 8 carbon atoms, or about 4 to about 6 carbon atoms. Additionally, the aliphatic amino acids may have a terminal amino group. Exemplary amino acids include glycine, β-alanine, γ-aminobutyric acid, 5-aminovaleric acid, 2-methoxy-α-alanine, 2,4-diaminobutyric acid, as well as combinations thereof.

The aliphatic amino acid may be substituted or unsubstituted at either one or more of the carbon atoms therein, and/or a nitrogen atom therein, the substituents being selected from the list presented herein (see, e.g., the list of potential “R” substituents herein). However, in one particular embodiment the aliphatic amino acid is unsubstituted, while in another the aliphatic amino acid has about 1 or 2 substituents thereon, which may be the same or different.

In this regard it is to be noted that, conveniently, in one embodiment a substituted aliphatic amino acid may be used in the synthesis of the compound, rather than modifying the amino acid after the compound is formed. Alternatively, a functional group may be present on the chain of the substituent, if necessary being appropriately protected during the course of the synthesis, the functional group then being available for use in the subsequent modification. In some embodiments, such a functional group could be selectively used for synthesis of different compounds, so as to provide for substitution at that site to produce products having unique properties associated with a particular application.

As indicated above, these amino acids may play a specific role in the compound. For example, the longer chain aliphatic amino acid may serve to provide for turns in the molecule and/or to close the molecule to form a ring. The shorter chain aliphatic amino acids may be employed to provide a shift for spacing in relation to the dsDNA sequence to be specifically or preferentially bound, and/or to provide enhanced binding by being present proximate a terminal cyclic or heterocyclic group. For purposes of space-shifting, glycine and alanine (e.g., β-alanine) are preferred for some embodiments.

The aliphatic amino acid may be present at one or both ends of the compound. In addition, in some embodiments a consecutive sequence of more than about 6, 8 or even 10 heterocycles is avoided by means of inserting an aliphatic amino acid. For example, an amino acid such as glycine or -alanine may be introduced in an otherwise consecutive series of about 6, 8 or even 10 cyclic or heterocyclic moieties; stated another way, in some embodiments the compound may comprise such an aliphatic amino acid bordered by at least about 2, about 3, about 4, about 5, etc. heterocyclic moieties.

It is to be noted that, for some embodiments, when an aliphatic amino acid is C-terminal, the carboxyl group may be functionalized as an amide or an ester, where the alcohol or amino acid may be selected, for example, to provide for specific properties or be used to reduce the charge of the carboxyl group. In the latter situation, the alcohol and amino groups may be, for example, from about 1 to about 6 carbon atoms, or from about 2 to about 4 carbon atoms.

E. Exemplary Compound Structures

In view of the foregoing, it is to be noted that, in some embodiments of the present invention, the compound of the present invention may have the general structure: 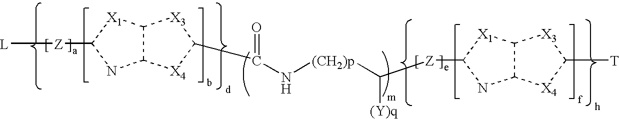
wherein:
L is independently selected from H, H₂N(HN)CNHCH₂ (wherein the terminal methylene group, CH₂, is attached to the carbonyl carbon, and further wherein one or both of the terminal amine nitrogen atoms may be positively charged), and a non-tautomerizing, fused bicyclic structure: 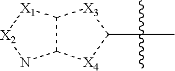
and further wherein each ring of each non-tautomerizing fused, bicyclic structure has 5-members or 6-members, provided both rings are not 5-member rings, and still further wherein, as represented by the dashed lines therein, at least one ring of each structure is heteroaromatic and the other is aromatic or heteroaromatic (i.e., the overall structure is heteroaromatic);
X₁ and X₂ are independently selected from O, S, N, NR², CR³, CR⁴═CR⁴¹, CR⁴═N, N═CR⁴, N═N and CR⁴, provided that (i) when each one of X₁ or X₂ is independently selected from O, S or NR², the other is independently selected from CR³ or N, and (ii) when each one of X₁ or X₂ is independently selected from CR⁴═CR⁴′, CR⁴═N, N═CR⁴ or N═N, the other is independently selected from CR⁴″, or N;
X₃ is independently selected from N, O, S, CR⁵, NR⁵, CR⁵═CR⁵′, CR⁵═N, N═CR⁵ and N═N, and X₄ is independently selected from O, S, N and CH, provided that (i) when each X₃ is independently selected from CR⁵ or N, X₄ is independently selected from O or S, and (ii) when each X₃ is independently selected from O, S, NR⁵, CR⁵═CR⁵′, CR⁵═N, N═CR⁵ or N═N, X₄ is independently selected from CH or N;
T is an amido-containing structure: 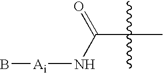
wherein A, when present, is independently selected from —CH₂CH₂C(O)— or —CH₂C(O)—, wherein the terminal methylene group is bound to nitrogen and the terminal carbonyl carbon is bound to B; and, B is independently selected from a diamine or triamine end-group, which may optionally be positively charged under physiological conditions known in the art;
Y, when present, is independently selected from H, NH₂, OH, SH, Br, Cl, F, OCH₃, CH₂OH, CH₂SH and CH₂NH₂, provided that, in some embodiments, when (i) Y is NH₂, p is about 2, and (ii) Y is OCH₃, p is about 1;
Z is independently selected from (i)—C(O)NH—Q—, wherein Q is independently selected from substituted or unsubstituted C₁₋₆ alkyl (e.g., methyl, ethyl, propyl, butyl, etc.), or (ii) one of structures (1), (2), (3) and (4): 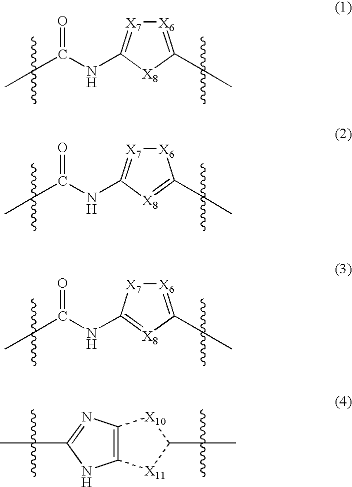
wherein (i) the bonds extending from the carbonyl carbon atom and the carbon atom adjacent X₆ (i.e., between X₆ and X₈) in structures (1), (2), and (3), as well as the bonds extending from the carbon atoms between the two nitrogen atoms and X₁₀/X₁₁ in structure (4), indicate sites of attachment to the remaining portion of the compound, and further wherein
   for structure (1) X₆ is CR⁶, X₇ is independently selected from CR⁷ or N, and X⁸ is independently selected from O or S,
   for structure (2))₆ is independently selected from NR⁶, O or S, X₇ is independently selected from CR⁷ or N, and X⁸ is independently selected from CH, C(OH), or N,
   for structure (3) X₆ is independently selected from CR⁶ or N, X₇ is independently selected from NR⁷, O or S, and X⁸ is independently selected from CH, C(OH), or N; and,
   for structure (4) each ring is unsaturated, X₁₀ is independently selected from CR¹⁰═CR¹⁰′, CR¹⁰═N, N═CR¹⁰ or N═N, and X¹⁰ is independently selected from CH, C(OH), or N;
each substituent R², R³, R⁴, R⁴′, R⁴″, R⁵, R⁵′, R⁶, R⁷, R¹⁰ and R¹⁰′ is independently selected from H, hydroxy, N-acetyl, benzyl, substituted or unsubstituted C₁₋₆ alkyl (e.g., methyl, ethyl, propyl, butyl, pentyl, hexyl), substituted or unsubstituted C₁₋₆ alkylamine (e.g., methylamine, ethylamine, propylamine, butylamine, pentylamine, hexylamine), substituted or unsubstituted C₁₋₆ alkyldiamine (e.g., methyldiamine, ethyldiamine, propyldiamine, butyldiamine, pentyldiamine, hexyldiamine), substituted or unsubstituted C₁₋₆ alkylcarboxylate (e.g., methylcarboxylate, ethylcarboxylate, propylcarboxylate, butylcarboxylate, pentylcarboxylate, hexylcarboxylate), substituted or unsubstituted C₂₋₆ alkenyl (e.g., ethenyl, propenyl, butenyl, pentenyl, hexenyl), substituted or unsubstituted C₂₋₆ alkynyl (e.g., ethynyl, propynyl, butynyl, pentynyl, hexynyl) and, when attached to a carbon atom, optionally halo (e.g., chloro, bromo, fluoro, iodo), provided that (i) when X¹ or X² is NR², R² is other than H, and (ii) when X³ is NR⁵, R⁵ is other than H; and,
subscripts a, b, d, e, f, h, i, and p are each, independently, greater than or equal to 0 (e.g., each of these is, independently, about 1, 2, 3, 4, 5 or more), and subscripts m and q are 0 or 1, provided that (i) when L is not a non-tautomerizing, fused, bicyclic structure, b or f is at least about 1 (e.g., each of these is, independently, about 1, 2, 3, 4, 5 or more), (ii) when m is 0, q and p are also 0; (iii) the result of [(a+b)*d] is at least about 2 (e.g., about 3, 4, 5 or more); and, (vi) the result of [(e+f)*h] is the same or different from the result of [(a+b)*d] and is greater than or equal to 0 (e.g., about 1, 2, 3, 4, 5 or more), further provided that when the result of [(e+f)*h] is 0, m is 0.

In this regard it is to be noted that the amino reagents commonly used to cleave polyamides from a resin following synthesis, and thus the reagents that may be used to remove the compounds of the present invention from a resin following synthesis using, for example, automated techniques known in the art (and further described herein below), include for example 3-(dimethylamino)-propylamine, ethylenediamine, and 3,3′-diamino-N-methyldipropylamine. Accordingly, B may in some embodiments be independently selected from a derivative of (CH₃)₂N(CH₂)₃NH₂, H₂NCH₂CH₂NH₂, and CH₃N(CH₂CH₂NH₂), respectively.

It is to be further noted that the above-described compound structure it intended to encompass numerous permutations. For example, with respect to those portions of the above-described compound associated with subscripts a, b, e and f, it is to be noted that these portions may generally be viewed as present or absent based on the respective values of these subscripts (e.g., wherein the value of “1” means that portion is present and the value of “0” means it is absent). As such, as the values of d and h independently become greater than 1, various combinations of a and b, and/or e and f, respectively, may be present in the compound as the values of a, b, e and f independently vary from 0 to 1 (given that, as d and/or h exceed 1, each portion associated with a, b, e and/or f, respectively, may be the same or different).

Accordingly, in some independent embodiments: (i) d may be less than or equal to about 8, 6, 4 or even about 2; (ii) h may be less than or equal to about 8, 6, 4 or even about 2; (iii) the result of (a+b)*d may be less than or equal to about 10, 8, 6 or even about 4; (iv) the result of (e+f)*h may be less than or equal to about 10, 8, 6 or even about 4; (v) i may be less than about 5, 4, 3 or even 2; and/or (vi) p may be less than about 10, 8, 6 or even 4. Furthermore, in some of these or other embodiments: L is the fused, bicyclic structure shown; d is about 1 or 2; and/or the result of (a+b)*d ranges from about 2 to 8, or about 4 to 6, wherein b is 0, about 1 or about 2. In these or other embodiments, additionally or optionally: m is about 1 or 2; p ranges from about 2 to 8, or about 4 to 6; and/or q is 0 or about 1. In these or still other embodiments, additionally or optionally: T is the amido-containing structure shown; h is about 1 or 2; and/or the result of (e+f)*h ranges from about 2 to 8, or about 4 to 6, wherein f is 0, about 1 or about 2. Additionally, in some embodiments the result of (e+f)*h is about the same as the result of (a+b)*d. Alternatively, in some embodiments h is 0.

In this regard it is to be noted that, in one preferred embodiment, L is the fused, bicyclic structure shown above and (i) Y is NH₂ and p is 2, or (ii) Y is OCH₃ and p is 1. In another preferred embodiment, L is the fused, bicyclic structure shown above and the result of a+b is in the range of about 1 to about 10, preferably about 2 to about 8, and more preferably about 4 to about 6. In yet another preferred embodiment, L is the fused, bicyclic structure shown above and the result of e+f is 0, and m is 0. In yet another preferred embodiment, L is the fused, bicyclic structure shown above, wherein X₁ is independently selected from N-methyl, S or O, X₂ is CH, X₃ is CH═CH, and X₄ is CH.

It is to be further noted that, in those instances wherein a mixture of compounds are present, the above-noted compositional numbers (i.e., the numbers which represent subscripts a, b, d, e, f, h, i, m, p, and q) may represent an average.

It is to be still further noted that when Z is a fused, bicyclic structure (e.g., structure (4), as illustrated above), in one embodiment this structure may be tautomerizing, such that it may act as a H-bond donor or H-bond acceptor, under physiological conditions.

Among the exemplary compounds of the present invention are those listed in Table 1 of Example 6, below (in particular the second through ninth compounds listed/illustrated therein). Additional exemplary compounds include those having the formula: 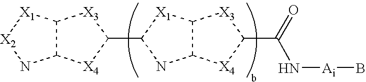
wherein X₁, X₂, X₃, X₄ (which may be the same or different for each fused, bicyclic structure present), as well as A, B, subscript i and subscript b, are as previously described. In these or other embodiments, the fused, bicyclic structure may be: 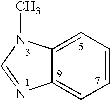
wherein, when (i) said fused bicycle occupies a first terminal position within the compound, carbon C7 forms a bond with the remaining portion of the compound (the 6-member ring being the second ring of the fused, bicyclic structure, or the ring closest to the tail-end of the compound), and (ii) said fused bicyclic structure occupies a non-terminal position within the compound, the heterocyclic ring thereof is the first ring (i.e., the ring closest to the cap or initial end of the compound), carbons C2 and C7 forming bonds with the remaining portion of the compound.

In other embodiments, the fused, bicyclic structure may be: 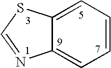
wherein, when (i) said fused bicycle occupies a first terminal position within the compound, carbon C7 forms a bond with the remaining portion of the compound (the 6-member ring being the second ring of the fused, bicyclic structure, or the ring closest to the tail-end of the compound), and (ii) said fused bicyclic structure occupies a non-terminal position within the compound, the heterocyclic ring thereof is the first ring (i.e., the ring closest to the cap or initial end of the compound), carbons C2 and C7 forming bonds with the remaining portion of the compound.

In still other embodiments, the fused, bicyclic structure may be: 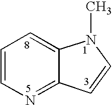
wherein, when (i) said fused bicycle occupies a first terminal position within the compound, carbon C2 forms a bond with the remaining portion of the compound (the 5-member ring being the second ring of the fused, bicyclic structure, or the ring closest to the tail-end of the compound), and (ii) said fused bicyclic structure occupies a non-terminal position within the compound, the heterocyclic ring thereof is the first ring (i.e., the ring closest to the cap or initial end of the compound), carbons C2 and C6 forming bonds with the remaining portion of the compound.

Additionally, in one or more of the above-described embodiments, at least one Z may have the structure: 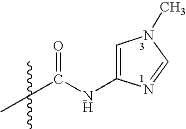
wherein (i) the non-substituted N atom (N1) is directed toward the floor of the minor groove, and (ii) carbon C2 and the carbonyl carbon form bonds with the compound when the moiety occupies an internal position therein. In this or other embodiments, at least one Z may also have the structure: 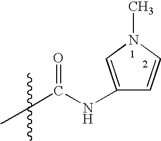
wherein (i) the substituted N atom is directed away from the floor of the minor groove, and (ii) carbon atom C2 and the carbonyl carbon form bonds with the compound when the moiety occupies an internal position therein.

Other exemplary embodiments may include, in view of the foregoing: 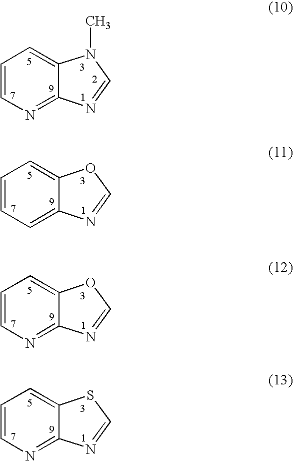
In this regard it is to be noted that structures (10), (12) and (13) may be oriented within, or connected to, the remaining compound in either direction; that is, for these structures, either ring may be the first ring (i.e., the ring which is farthest from the tail or end of the compound).

In still other exemplary embodiments, the compound of the present invention may additionally include a fused, bicyclic structure which acts as a H-bond donor, a heteroatom therein thus having a hydrogen substituent attached thereto (or being capable of forming a tautomer under physiological conditions, such that a hydrogen atom is attached thereto). For example, such compounds may have a structure such as: 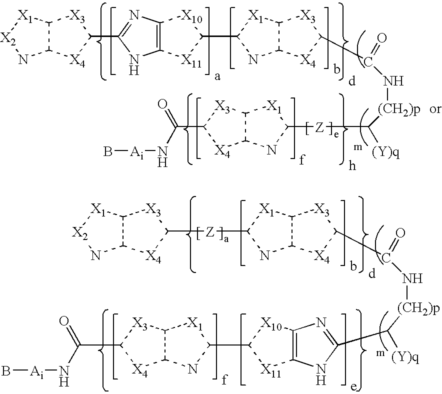
wherein L and T are as shown, Z is a fused, bicyclic structure, which may be the same or different, in one or more locations within the compound, and each designation or variable (e.g., X₁, X₂, X₃, X₄, X₉, X₁₀, X₁₁, etc.) is as defined previously.

In this regard it is to be noted that although only one “leg” of the above compounds is shown containing a Z moiety that is as a fused, bicyclic structure, each leg of the compound may alternatively contain such a moiety, or more than one of such moieties. Additionally, when more that one of such moieties is present, they may be the same or different without departing from the scope of the present invention.

In those instances wherein compounds (e.g., analogs of polyamide oligomers or polymers) are prepared using the fused, bicyclic structure of the present invention (as a cap and/or a aa non-terminal moiety), as well as for example N-methylpyrrole and N-methyl imidazole, the following are additional examples of compounds of the present invention (wherein “Py” refers to N-methylpyrrole, “Im” refers to N-methyl imidazole, “Cap” refers to a fused, bicyclic structure of the present invention in an initial, terminal position within the oligomer, “FBS” refers to a non-tautomerizing, fused, bicyclic structure in a non-terminal position within the oligomer, which may be different from, or essentially the same as (differing only at the second point of attachment), the “Cap”; “γ” is γ-aminobutyric acid, and, “Im/FBS” refers to a moiety of the oligomer which may be either N-methyl imidazole or a non-tautomerizing, fused, bicyclic structure).

| | |
|---|---|
| CapPyPyPy-γ-PyPyPyPy, | PyPyFBSPy-γ-PyPyPyPy, |
| CapPyPyPy-γ-Im/FBSPyPyPy, | PyFBSPyPy-γ-PyIm/FBSPyPy, |
| PyIm/FBSPyPy-γ-PyImPyPy, | CapPyIm/FBSPy-γ-PyPyPyPy, |
| CapIm/FBSPyPy-γ-PyPyPyPy, | Im/CapIm/FBSPy-γ-PyPyPyPy, |
| CapIm/FBSPyPy-γ-ImFBSPyPyPy, | ImPyPyPy-γ-Im/FBSIm/FBSPyPy, |
| Im/CapIm/FBSPyPy-γ-ImImPyPy, | Im/CapPyImPy-γ-Im/FBSPyImPy, |
| CapIm/FBSImPy-γ-ImPyPyPyPy, | CapIm/FBSIm/FBSIm-γ-PyPyPyPy, |
| Im/Cap-β-PyPy-γ-Im/FBS-β-PyPy, | Im/Cap-β-Im/FBSIm-γ-Py-β-PyPy, |
| Cap-β-ImPy-γ-Im-β-Im/FBSPy, | CapPyPyPyPy-γ-Im/FBSPyPyPyPy, |
| ImIm/FBSPyPyPy-γ-ImPyPyPypy, | ImPyIm/FBSPyPy-γ-ImPyPyPyPy, |
| ImImPyImFBSIm-γ-PyPyPyPyPy, | Im/CapPyPyImPy-γ-ImPyPyImPy, |
| Cap/ImPy-β-PyPy-γ-ImPy-β-PyPy, | ImIm-β-ImIm/FBS-γ-PyPy-β-PyPy, |
| ImPy-β-Im/FBSPy-γ-ImPy-β-ImPy, | CapPy-β-PyPyPy-γ-ImPyPy-β-PyPy, |

CapIm-β-PyPyPy-γ-PyPyPy-β-PyPy,
CapIm/FBSPy-β-Im/FBSPyPy-γ-Im/FBSPyPy-β-PyPy,
CapFBS-β-PyPyPy-γ-Im/FBSIm/FBSPy-β-PyPy,
CapPy-β-PyPyPy-γ-PyPyPy-β-Im/FBSPy,
CapPyPyPyPyPy-γ-Im/FBSPyPyPyPyPy,
CapPyPy-β-PyPy-γ-Im/FBSPyPy-β-PyPy,
ImPyPyPy-β-Py-γ-Im/FBS-β-PyPyPyPy,
CapIm/FBSPyPyPyPy-γ-Im/FBSIm/FBSPyPyPyPy,
Cap-β-PyPyPyPy-γ-Im/FBS-β-PyPyPyPy,
Im/FBSPyPyPy-β-Py-γ-Im/FBSPyPyPy-β-Py,
CapPyIm/FBSPyPyPy-γ-Im/FBSPyPyPyPyPy,
CapPyPy-β-PyPy-γ-Im/FBSPy-β-PyPyPy,
Cap/Im-β-PyPyPyPy-γ-FBSPyPyPy-β-Py,
Cap-β-Im/FBSPyPyPy-γ-Im/FBSPyPyPy-β-Py,
CapIm/FBS-β-PyPy-β-PyPy-β-PyPy,
CapIm-β-Im/FBSIm/FBSIm/FBSIm/FBS-γ-PyPyPyPy-β-Py,
CapPy-β-Im/FBSPy-β-Im/FBSPy-γ-Im/FBSPy-γ-Im/FBSPy-β-Im/FBSPy,
Cap/ImPy-β-PyPy-β-PyPy-γ-Im/FBSPy-β-PyPy-β-PyPy,
and
Im-β-Im/FBSIm/FBSIm/FBSIm/FBS-β-Im/FBS-γ-Py-β-PyPyPyPy-β-Py.

In this regard it is to be noted that the above compounds are simply exemplary and, therefore, the above list should not be consider limiting. For example, generally speaking, the fused, bicyclic structure of the present invention may be similarly employed in essentially any polyamide, or analog thereof, known in the art.

Finally, it is to be noted that one or more of the compounds described herein may further comprise, for example, a linker suitable for attaching it to, for example, a support, a peptide, a sugar, etc. The linker may be, for example, an aliphatic amino acid moiety or derivative, such as an ethylene glycol moiety (i.e., a moiety derived from ethylene glycol).

F. Size

It is to be noted that, generally speaking, the size of the compound of the present invention may be controlled in order to optimize, for example, binding affinity and/or selectivity for a given application, such as for example when they are to be used with cells (e.g., viable cells). Accordingly, in some embodiments the size may be less than about 25 kD, 20 kD, 15 kD, or even 10 kD, while in other embodiments the size may be less than about 5 kD, 4 kD, 3 kD, 2 kD, 1 kD or even 0.5 kD. Further, in some embodiments size may range from about 0.5 kD to about 20 kD, or from about 1 kD to about 10 kD.

However, it is to be noted in this regard that, depending upon the particular application, the compound of the present invention may include various additional groups (as further illustrated, for example, by the discussion below). As a result, it is to be understood that the final size may vary and thus may be other than herein described without departing from the scope of the present invention.

G. Optional End or Tail Group

Optionally, the tail end or terminal of the compound may have a group which alters one or more properties of the compound for an intended purpose. For example, as further described in, for example, U.S. Pat. No. 6,303,312, which is incorporated herein by reference:
1. A polar substituent may be present on an alkyl group, where the polar group may be from about 2 to about 6, or about 3 to about 4, carbon atoms from the linkage to the remaining molecule. The polar group may be charged or uncharged, where the charge may be, for example, a result of protonation under the conditions of use. Particularly, groups capable of hydrogen bonding, such as an amino group (e.g., tertiary-amino), hydroxyl, mercapto, and the like, may be employed. Of particular interest in one embodiment is amino, more particularly alkylated amino, where the alkyl groups include from about 1 to about 6, or about 2 to about 4, carbon atoms, wherein at a pH less than about 8 the amino group is positively charged and can hydrogen bond with the dsDNA. In at least one embodiment, 2 positively charged polar groups are not employed such that they are in juxtaposition when complexed with the dsDNA because, without being held to a particular theory, it is believed that this may act to reduce the binding affinity of the oligomer.
2. An isotopic group may be present to enable oligomer detection using scintillation counters for radioactive elements, NMR for atoms having a magnetic moment, and the like. For a radioactive oligomer, a radioactive label may be employed, such as tritium, ¹⁴C, ¹²⁵I, or the like. Such a label may serve numerous purposes in, for example, diagnostics, cytohistology, radiotherapy, and the like.
3. Additionally, in diagnostic applications, for example, one may wish to have a detectable label other than a radiolabel. The oligomer may therefore be linked to labels which are fluorescent (e.g. dansyl, BODIPY, fluorescein, Texas red, isosulfan blue, ethyl red, malachite green, etc.), which exhibit chemiluminescence, which are light sensitive (e.g., bond forming compounds such as psoralens, anthranilic acid, pyrene, anthracene, and acridine), etc.
4. Alternatively, the lipophilicity of the oligomer may desirably be enhanced by the addition of a lipophilic group, such as cholesterol, a fatty acid, a fatty alcohol, a sphingomyelin, a cerebroside, and the like, where the fatty group will generally be from about 6 to about 30 carbon atoms, or about 8 to about 25 carbon atoms, or about 10 to about 20 carbon atoms.
5. The compound may also include, for example, a saccharide (which bind to lectins, adhesion molecules, bacteria or the like), where the saccharide serve to direct the subject oligomer to a specific cellular target.

The different molecules may be joined to the termini of the compound (or, in these or other embodiments, to a non-terminal position within the compound) in a variety of ways known in the art, using means known in the art. For example, such molecules may be introduced as part of the synthetic scheme, displacing the compound from the solid support on which it was synthesized.

In this regard it is to be noted that the above list is not intended to be exhaustive and, as such, should not be viewed to limit the scope of the present invention. In generally, the compounds of the present invention may be employed with essentially any additional substituent or tail group known in the art. For example, the compounds may additionally comprise an end or tail group such as a DNA cleavage agent, or some other binding agent such as an oligonucleotide or peptide.

H. Compound Preparation

The subject compounds may be synthesized using means known in the art (see, e.g., U.S. Pat. Nos. 6,090,947 and 6,303,312 which are incorporated herein by reference). For example, as further illustrated in the Examples provided herein below, they may be prepared on supports (e.g. chips) using automated synthetic techniques known in the art (see, e.g., J. Am. Chem. Soc., 118, 6141 (1996)). For example, the compound may be grown on a solid phase, being attached to a solid support by a linkage which can be cleaved by a single step process. The addition of an aliphatic amino acid at the C-terminus of the compounds allows the use of, for example, Boc-β-alanine-Pam-resin, which is commercially available in appropriate substitution levels (e.g., 0.2 mmol/g). Aminolysis may be used for cleaving the compound from the support. In the case of the N-methyl-4-amino-2-carboxypyrrole and the N-methyl-4-amino-2-carboxyimidazole, the t-butyl esters may be employed, with the amino groups protected by Boc or Fmoc, with the monomers (i.e., building blocks or moieties of the compound) added sequentially in accordance with conventional techniques.

In some instances, different compounds may be synthesized at individual sites on a single substrate (e.g., about 10, about 25, about 50, about 75, about 100, about 500, about 1000 or more). In this way, an array of different compounds may be synthesized, which can then be used to identify the presence of a plurality of different nucleotide sequences in a sample. By knowing the composition of the compound at each site, one can identify binding of specific sequences at that site by various techniques, such as labeled anti-DNA antibodies, linkers having complementary restriction overhangs, where the sample DNA has been digested with a restriction enzyme, and the like. The techniques for preparing the subject arrays are analogous to the techniques used for preparing oligopeptide arrays known in the art (see, e.g., Cho et al., Science, 1993, 261, 1303-1305).

III. dsDNA Binding

A. The Compound/dsDNA Triplex

It is to be noted that the present invention enables the preparation of compounds (e.g., analogs of polyamide oligomers or polymers) which will bind with nucleotide sequences, containing at least 1 guanine nucleotide therein, with specificity. In some embodiments, a single compound/dsDNA triplex may bind to form a single entity or species, while in other embodiments combinations of compounds of the present invention (e.g., about 2, about 4, about 6, about 8, about 10 or more, the compounds being the same, substantially the same, or different) may be utilized to bind the dsDNA, in order to form such a triplex. In part, the number of compounds utilized may depend, in some embodiments, upon whether there is a hairpin turn therein. If such a turn is present, a single compound may be utilized, whereas when such a turn is not present multiple compounds may be needed in order for sufficient complementation to be achieved. Additionally, multiple compounds (e.g., hairpin-containing or non-hairpin-containing compounds, or a combination thereof) may be used, for example, when more than one target sequence of dsDNA is present for binding (e.g., contiguous or proximate target sequences, in order to enhance the overall binding specificity, or distal sequences, wherein the sequences may be associated with the same functional unit (e.g., a gene) or different functional units (e.g., homeodomains)).

In this regard it is to be noted that, as used herein, “triplex” generally refers to the species which results from a dsDNA and the compound(s) of the present invention becoming bound together by H-bonding, and optionally other interactions, in the minor groove of the double strand, wherein the non-tautomerizing, fused, bicyclic structure is in registry with, and H-bonds to, a G nucleotide as an acceptor.

It is to be further noted that, when a turn (e.g., hairpin- or γ-) is present, each portion of the compound before and after the turn may be referred to, for example, as a “leg” of the hairpin, or tandem, unit. Each “leg” may, for example, independently comprise about 2 to about 10, or about 4 to about 8, moieties selected from, for example: a fused, bicyclic structure; a non-fused, non-bicyclic heteroaromatic moiety; or, an aliphatic amino acid, all as described elsewhere herein. Additionally, it is to be noted that each leg may comprise a different number of such moieties. In one preferred embodiment, the compound of the present invention additionally comprises a fused, bicyclic structure which acts as a H-bond donor moiety, said structure having a heteroatom therein which is hydrogen substituted.

It is to be still further noted that, in one embodiment, the compound of the present invention may comprise multiple hairpin, or tandem, units, each of said units being linked, for example, at one position by the aliphatic amino acid therein which enables the hairpin turn therein. Such compounds may comprises, for example, at least about 2 hairpin or tandem units (e.g., at least about 4, 6, 8, 10, 25, 50, 75 or even 100), the number of units present therein ranging, for example, from about 2 to about 100, from about 4 to about 75, from about 6 to about 50, or from about 8 to about 25 units.

The compound/dsDNA triplex, whether a single compound or a combination of compounds are present, in some embodiments comprises at least about 2, about 4, about 6, about 8, about 10 or more complementary base pairs. Further, in these or other embodiments, not more than about 50, about 40, about 30, or even about 20 complementary base pairs are present. Additionally, the orientation of the compound, in some embodiments, is amino to carbonyl (or “N to C”) in association with the 5′ to 3′ direction of the strand to which it is juxtaposed or bound.

Accordingly, it is to be noted that the compound, or polyamide analog, may have at least about 3, about 4, about 5, about 6 or more consecutive pairs comprising carboxamides and fused, bicyclic structures, for binding with specificity a sequence of nucleotides having at least about 3, about 4, about 5, about 6 or more, respectively, DNA base pairs, in the minor groove of the dsDNA, said sequence having at least about one A/T or T/A DNA base pair and at least about one G/C or C/G base pair. In one preferred embodiment, the sequence of dsDNA is a regulatory sequence, a promoter sequence, a coding sequence, or a non-coding sequence.

It is to be further noted that, in some embodiments wherein 2 or more compounds are used, the compound pairs may be completely overlapped, or only partially overlapped (i.e. slipped or having overhangs). In the overlapped configuration, the heterocyclic rings(e.g., azoles rings such as N-methylpyrrole, imidazole and/or the fused, bicyclic structure) may be in complementary pairs, as well as any spacing amino acid moiety or linker. In the slipped configuration, there may be in some embodiments at least about 1 ring which is unpaired in at least about 1 of the compounds, and usually there may be at least about 2 rings or more (e.g., 4, 6, 8, 10, etc.) in both of the compounds. The number of unpaired rings may be, in some embodiments, in the range of about 2 to about 40, about 4 to about 20, or about 5 to about 10. In some embodiments, unpaired rings may involve chains of about 2 or more rings, or even about 3, 4, or more rings, including, as appropriate, aliphatic amino acids in the chain.

It is to be still further noted that the triplex described herein may be part of a cell; that is, a cell may comprise the triplex, said cell being, for example, eukaryotic (e.g., a mammalian cell), or prokaryotic (e.g., a bacteria).

It is to be understood, in view of the foregoing, that various permutations and combinations of compounds/dsDNA triplex may be prepared and used herein, without departing from the scope of the invention.

B. Affinity/Selectivity

In some embodiments of the present invention, the compound/dsDNA triplex includes a compound having a non-tautomerizing, fused, bicyclic structure as a cap, and optionally one or more of such structures, which may be the same or different, occupying non-terminal positions therein. Additionally, the compound comprises one or more heterocycles, such as pyridine, and optionally imidazole (e.g., N-methyl imidazole). Further, because the compound comprises moieties that have specificity for one nucleotide, which are thus present in the triplex as a complementary pair, it is to be noted that in some embodiments the subject triplexes will accordingly have at least one of these complementary pairs, and frequently at least about 2, 4, 6, 8, 10 or more of these complementary pairs. In at least some instances, however, generally fewer than about 85%, 75%, 65% or even 50% of the complementary pairs in the complex will have such specificity (i.e., less than about 85%, 75%, 65%, or even 50% of the complementary pairs will include a fused, bicyclic structure or an imidazole).

Additionally, while in some embodiments there is at least one complementary pair involving a fused, bicyclic structure, in these or other embodiments there may be less than about 10, 8, 6, 4 or even 2 of such pairs, and/or complementary pairs involving a fused, bicyclic structure and/or an imidazole consecutively, so that there are no more than about 10, 8, 6, 4 or even 2 fused, bicyclic structures and/or imidazoles in a row within the compound. Accordingly, in these or other embodiments there may be, for example, at least about 1 to about 10, about 2 to about 8, or about 4 to about 6 complementary pairs present, which may or may not have about 2 or more consecutive pairs involving a fused, bicyclic structure and/or an imidazole. Therefore, in these or other embodiments the compound of the present invention may additionally comprise at least about 1, 2, 3, 4, 5 or more aliphatic amino acids in the compound. Alternatively, in these or other embodiments the compound may comprise less than about 10, about 8, about 6, or even about 4 aliphatic amino acids therein. In some embodiments, there may be an amino acid proximate at least one terminus (e.g., the tail) of the compound.

In this regard it is to be noted that, without being held to a particular theory, the number of fused, bicyclic structures and/or imidazoles present in the compound may, in some embodiments, be limited because while these add greater specificity, they may contribute less than other compound moieties (e.g., heterocycles such as N-methylpyrrole) to the binding affinity for the dsDNA. Accordingly, for a given target sequence, binding affinity and specificity may be optimized by appropriate selection of compound components or moieties. For example, in some instance the compound may have a binding affinity, Kₐ (as determined using means known in the art, such as DNase I footprint analysis; see, e.g., the Experimental section of U.S. Pat. No. 6,303,312 and/or PCT Application No. WO 02/34295, which are incorporated herein for this and all other relevant purposes), that is greater than about 1×10⁶ M⁻¹, about 1×10⁷ M⁻¹, about 1×10⁸ M⁻¹, about 1×10⁹ M⁻¹, about 1×10¹⁰ M⁻¹, about 1×10¹¹ M⁻¹, or even about 1×10¹² M⁻¹, so as to be able to bind to the target sequence at submicromolar concentrations or less (e.g., nanomolar or picomolar) in the environment in which they are used.

In comparison, with respect to selectivity, it is to be noted that the difference in affinity with a single mismatch may be at least about 2 fold, about 3 fold, about 5 fold, about 10 fold, about 25 fold, about 50 fold, about 75 fold, about 100 fold, or more; that is, the compound is about 2, about 3, about 5, about 10, about 25, about 50, about 75, about 100 or more times likely to bind a “target” nucleotide sequence over a mismatch nucleotide sequence. Stated another way, the ratio of the binding affinity of the compound or polyamide analog of the present invention with the sequence that is to be bound with specificity, as compared to the association constant of the compound with a sequence that is not to be so bound, may be at least about 2 times, about 3 times, about 5 times, about 10 times, about 25 times, about 50 times, about 75 times, or even about 100 times greater.

Additionally, it is to be noted that the triplex of the present invention may have a dissociation constant of no more than about 50, about 40, about 30, about 20, about 10, about 5, about 1, about 0.5, or even about 0.1 nanomolar or less, as determined by means standard in the art.

C. Compound/dsDNA Triplex Preparation and Use

The compound of the present invention may be brought together with a sequence of oligonucleotides, at least one of which is a guanine nucleotide, in a minor groove of dsDNA under a variety of conditions known in the art, using a variety of techniques known in the art, for a variety of different purposes known in the art. For example, the conditions under which a compound/dsDNA triplex is formed may be in vitro, in cell cultures, ex vivo or in vivo. For purposes of detecting the presence of a target sequence, the dsDNA may be extracellular or intracellular. When extracellular, the dsDNA may be in solution, in a gel, on a slide, or the like. The dsDNA may also be part of an episomal element. Finally, the dsDNA may be present as smaller fragments ranging from at least about 25, at least about 50, at least about 75, or at least about 100 base pairs, up to about 500, 1000, 2500, 5000 or more (e.g. several thousands, tens of thousands, or even a million base pairs or more); stated another way, the dsDNA fragment may range in size, for example, from about 25 to about 5000 base pairs, from about 50 to about 2500 base pairs, from about 75 to about 1000 base pairs, or from about 100 to about 500 base pairs. The dsDNA may be intracellular, chromosomal, mitochondrial, plastid, kinetoplastid, or the like, part of a lysate, a chromosomal spread, fractionated in gel electrophoresis, a plasmid, or the like, being an intact or fragmented moiety.

The formation of triplexes between dsDNA and the present compounds may be for diagnostic, therapeutic, purification, or research purposes, and the like. Because of the specificity of the compounds of the present invention, they may be used to detect specific dsDNA sequences in a sample, for example without melting of the dsDNA. The diagnostic purpose for the triplex formation may be, for example, detection of alleles, identification of mutations, identification of a particular host (e.g. bacterial strain or virus), identification of the presence of a particular DNA rearrangement, identification of the presence of a particular gene (e.g. multiple resistance gene, forensic medicine, or the like). With pathogens, the pathogens may be viruses, bacteria, fungi, protista, chlamydia, or the like. With higher hosts, the hosts may be vertebrates or invertebrates, including insects, fish, birds, mammals, and the like or members of the plant kingdom.

When involved in vitro or ex vivo, the dsDNA may be combined with the subject compounds in appropriately buffered medium, generally at a concentration in the range of about 0.1 nM to 1 mM. Various buffers may be employed, such as TRIS, HEPES, phosphate, carbonate, or the like, the particular buffer not being critical to this invention. Generally, conventional concentrations of buffer will be employed, usually in the range of about 10 to about 200 mM. Other additives which may be present in conventional amounts include sodium chloride, generally from about 1 to about 250 mM, dithiothreitol, and the like. The pH will generally be in the range of about 6.5 to 9. The target dsDNA may be present, for example, in an amount equal to about 0.001 to about 100 times the moles of compound.

The subject compounds, when used in diagnosis, may have a variety of labels (as indicated previously), and may use many of the protocols that have been used for detection of haptens and receptors (immunoassays) or with hybridization (DNA complementation), as known in the art. Since the subject compounds are not nucleic acids, it is generally believed that they can be employed more flexibly than when using DNA complementation. The assays may be carried out using methods known in the art and then, depending on the nature of the label and protocol, the determination of the presence and amount of the sequence may then be made. The protocols may be performed in solution or in association with a solid phase. The solid phase may be a vessel wall, a particle, fiber, film, sheet, or the like, where the solid phase may be comprised of a wide variety of materials, including gels, paper, glass, plastic, metals, ceramics, etc. Either the sample or the subject compound may be affixed to the solid phase in accordance with known techniques. By appropriate functionalization of the subject compounds and the solid phase, the subject compounds may be covalently bound to the solid phase. The sample may be covalently or non-covalently bound to the solid phase, in accordance with the nature of the solid phase. The solid phase allows for a separation step, which allows for detection of the signal from the label in the absence of unbound label.

Accordingly, a process detecting a nucleotide sequence of a dsDNA in a sample may comprise, for example, contacting, under triplex-forming conditions, a sample of dsDNA having a nucleotide sequence which comprises one or more guanine nucleotides a compound, or polyamide analog, of the present invention and further comprising a moiety for detecting triplex formation, and then detecting the presence of the dsDNA in the sample as a triplex with the compound by means of the detectable moiety. The detectable moiety may be, for example, an enzyme, a solid surface, a hapten which binds to a receptor, a radioactive isotope, or some other moiety that is detectable by means of fluorescence or chemiluminescence. The process may optionally further comprise separating the triplex from other dsDNA sequences present in the sample prior to triplex detection. In a preferred embodiment, the compound, or polyamide analog, is selected to provide an affinity K_{D} (wherein K_{D} is the product of an dissociation value (k_{d}) divided by an association value (kₐ), as determined by means known in the art) of less than about 50 nM (e.g., less than about 40 nM, about 30 nM, about 20 nM, about 10 nM, about 5 nM, about 1 nM, about 0.5 nM, or even about 0.1 nM).

Exemplary protocols include combining a cellular lysate, with the DNA bound to the surface of a solid phase, with an enzyme labeled compound, incubating for sufficient time under complex or triplex forming conditions for the compound to bind to any target sequence present on the solid phase, separating the liquid medium and washing, and then detecting the presence of the enzyme on the solid phase by use of a detectable substrate.

A number of protocols are based on having a label which does not give a detectable signal directly, but relies on non-covalent binding with a receptor, which is bound to a surface or labeled with a directly detectable label. In one assay one could have a hapten (e.g. digoxin) bonded to the compound. The sample DNA is bound to a surface, so as to remain bound to the surface during the assay process. The compound is then added and binds to any target sequence present therein. After washing to remove any unbound compound, an enzyme or a fluorescent labeled antidigoxin monoclonal antibody is added, the surface washed and the label detected. Alternatively, one may have a fluorescent tag or label bound to one end of the subject compound and biotin or other appropriate hapten bound to the other end thereof (or to its complement). These are combined with the DNA in the liquid phase and incubated. After completion of the incubation, the sample is combined with the receptor for the biotin or hapten (e.g., avidin or antibody, bound to a solid surface). After a second incubation, the surface is washed and the level of fluorescence determined.

If one wishes to avoid a separation step, one may use channeling or fluorescence quenching. By having two labels which interact, for example, two enzymes, where the product of one enzyme is the substrate of the other enzyme, or two compounds which fluoresce, where there can be energy transfer between the two compounds which fluoresce, one can determine when complex formation occurs, since the two labels will be brought into juxtaposition by forming the 2:1 complex in the minor groove. With the two enzymes, one detects the product of the second enzyme and with the two compounds which fluoresce, one can determine fluorescence at the wavelength of the Stokes shift or reduction in fluorescence of the fluorescent compound absorbing light at the lower wavelength. Another protocol would provide for binding the subject compound to a solid phase and combining the bound compound with DNA in solution. After the necessary incubations and washings, one could add labeled anti-DNA to the solid phase and determine the amount of label bound to the solid phase.

To determine a number of different sequences simultaneously or just a single sequence, one may provide an array of the subject compounds bound to a surface. In this way specific sites in the array will be associated with specific DNA sequences. One adds the DNA containing sample to the array and incubates. DNA which contains the complementary sequence to the subject compound at a particular site will bind to the compound at that site. After washing, one then detects the presence of DNA at particular sites (e.g., with an anti-DNA antibody, indicating the presence of the target sequence). By cleaving the DNA with a restriction enzyme in the presence of a large amount of labeled linker, followed by inactivation of the enzyme, one may then ligate the linker to the termini of the DNA fragments and proceed as described above. The presence of the label at a particular site in the array will indicate the presence of the target sequence for that site.

A number of protocols suitable for the present invention are known. (See, e.g., illustrative protocols for DNA assays in PCT Application Nos. WO 95/20591 and WO 86/05519, as well as European Application Nos. EP A393743 and EP A278220, while protocols and labels which may be adapted from immunoassays for use with the subject compound for assays for DNA may be found in, for example, PCT Application Nos. WO 96/20218; WO 95/06115; WO94/04538; WO94/01776; WO92/14490; EP A537830; WO91/09141; WO91/06857; and, WO91/05257.)

During diagnostics, such as involved with cells, one may need to remove the non-specifically bound compounds. This can be achieved by combining the cells with a substantial excess of the target sequence, conveniently attached to particles. By allowing for the non-specifically bound compounds to move to the extracellular medium, the compounds will become bound to the particles, which may then be readily removed. If desired, one may take samples of cells over time and plot the rate of change of loss of the label with time. Once the amount of label becomes stabilized, one can relate this value to the presence of the target sequence. Other techniques may also be used to reduce false positive results.

The subject compounds may also be used to titrate repeats, where there is a substantial change, increase or decrease, in the number of repeats associated with a particular indication. The number of repeats may be, for some embodiments, at least an increase of 50%, preferably at least two-fold, more preferably at least three-fold. By determining the number of compounds which become bound to the dsDNA, one can determine the amplification or loss of a particular repeat sequence.

The subject compounds may be used for isolation and/or purification of target DNA comprising the target sequence; that is, the subject compounds, or polyamide analogs, may be employed in a process of separating a nucleotide sequence of a dsDNA in a mixture of dsDNA. Generally speaking, such a process may comprise contacting, under triplex-forming conditions, a mixture of dsDNA nucleotide sequences and a compound, or polyamide analog, as set forth herein, wherein the compound or polyamide analog is suitable for binding a particular nucleotide sequence in said mixture with specificity, said compound or polyamide analog further comprising a moiety (e.g., a hapten) for separating said triplex once formed, and the separating the triplex formed between said dsDNA sequence and said compound, or polyamide analog, using said separation moiety. Optionally, the compound, or polyamide analog, and the dsDNA mixture are combined with a receptor for, as an example, a hapten bound to a solid surface. Preferably, the compound, or polyamide analog, is selected to provide an affinity K_{D} (wherein K_{D} is the product of a dissociation value (k_{d}) divided by an association value (kₐ), as determined by means known in the art) of less than about 50 nM (e.g., less than about 40 nM, about 30 nM, about 20 nM, about 10 nM, about 5 nM, about 1 nM, about 0.5 nM, or even about 0.1 nM).

By using the subject compounds, where the compounds are bound for example to a solid phase, those portions of a DNA sample which have the target sequence will be bound to the subject compounds and thus will be separated from the remaining DNA. One can prepare columns of particles to which the compounds are attached and pass the sample through the column. After washing the column, one can release the DNA which is specifically bound to the column using solvents or high salt solutions. Alternatively, one can mix particles to which the compounds are bound with the sample and then separate the particles, for example, with magnetic particles, using a magnetic field, with non-magnetic particles, using centrifugation. In this way, one can rapidly isolate a target DNA sequence of interest, for example, a gene comprising an expressed sequence tag (EST), a transcription regulatory sequence to which a transcription factor binds, a gene for which a fragment is known, and the like. As partial sequences are defined by a variety of techniques, the subject compounds allow for isolation of restriction fragments, which can be separated on a gel and then sequenced. In this way the gene may be rapidly isolated and its sequence determined. As will be discussed below, the subject compounds may then be used to define or alter the function of the gene.

The subject compounds may be used in a variety of ways, including for example in research and in methods of treatment. For example, these compounds or polyamide analogs may be used in a composition for regulating transcription which comprises a pharmaceutically acceptable excipient, and a transcription-regulating amount of the synthetic and/or non-naturally occurring compound, or polyamide analog, as set forth herein. Since these compounds, or more generally compositions comprising these compounds, can be used in a method to regulate (e.g., inhibit) transcription of a gene in a cell of an organism, the effect of regulating transcription on cells, cell assemblies and whole organisms may be investigated.

Generally speaking, a process for regulating transcription of a gene in a cell of an organism may comprise administering to the organism, or cell (e.g., a cultured cell), a transcription-regulating amount (e.g., an amount is in the range of about 0.1 nanomolar to about 1 millimolar, or about 10 nanomolar to about 1 micromolar) of one or more of the compounds or polyamide analogs set forth herein, or in a composition comprising one or more of the compounds or polyamide analogs as set forth herein. Such a process may be used to inhibit transcription, in for example, a gene of an organism (e.g., a mammal) or cell (e.g., a eukaryotic cell, such as a mammalian cell, or a prokaryotic cell, such as a bacterial cell). Additionally, the target dsDNA may be viral dsDNA. Optionally, the compound or polyamide analog may comprise a non-fused, non-bicyclic moiety capable of forming a hydrogen bond with a A, C or T nucleotide of said nucleotide sequence, either directly or by means of a H-bond donor linkage attached thereto.

Such a process may be performed in vitro, or in vivo. Additionally, such a process may be conducted in conjunction with egg cells, fertilized egg cells or blastocysts, to regulate (e.g., inhibit) transcription and expression of particular genes associated with development of the fetus, so that one can identify the effect of reduction in expression of the particular gene. Where the gene may be involved in regulation of a number of other genes, one can define the effect of the absence of such gene on various aspects of the development of the fetus. The subject compounds can be designed to bind to homeodomains, so that the transcription of one or more genes may be regulated (e.g., inhibited). In addition, one can use the subject compounds during various periods during the development of the fetus to identify whether the gene is being expressed and what the effect is of the gene at the particular stage of development.

With single cell organisms, one can determine the effect of the lack of a particular expression product on the virulence of the organism, the development of the organism, the proliferation of the organism, and the like. In this way, one can determine targets for drugs to regulate (e.g., inhibit) the growth and infectiousness of the organism.

In an animal model, one can provide for regulation (e.g., inhibition) of expression or over-expression of particular genes (e.g., oncogenes), reversibly or irreversibly, by administering the compound, or more generally the composition comprising the compound, to the host in a variety of ways (e.g., oral or parenteral, by injection, at a particular site where one wishes to influence the transcription, intravascularly, subcutaneously, or the like). By regulating (e.g., inhibiting) transcription, one can provide, for example, a reversible “knock out,” where by providing for continuous intravenous administration, one can greatly extend the period in which the transcription of the gene is regulated (e.g., inhibited). Alternatively, one may use a bolus of the subject oligomers and watch the effect on various physiological parameters as the bolus becomes dissipated. One can monitor the decay of the effect of the regulation (e.g., inhibition), gaining insight into the length of time the effect lasts, the physiological processes involved with the regulation and the rate at which the normal physiological response occurs. Instead, one can provide for covalent bonding of the oligomer to the target site, using alkylating agents, light activated bonding groups, intercalating groups, etc.

It is also possible to upregulate genes, by downregulating other genes. In those instances where one expression product inhibits the expression of another expression product, by inhibiting the expression of the first product, one can enhance the expression of the second product. Similarly, transcription factors involve a variety of cofactors to form a complex or triplex, one can enhance complex or triplex formation with one transcription factor, as against another transcription factor, by inhibiting expression of the other transcription factor. In this way one can change the nature of the proteins being expressed, by changing the regulatory environment in the cell.

It is to be noted that the target sequence may be associated with the 5′-untranslated region, namely the transcriptional initiation region, an enhancer, which may be in the 5′-untranslated region, the coding sequence or introns, the coding region, including introns and exons, the 3′-untranslated region, or distal from the gene.

The subject compounds may, in some embodiments, be presented as liposomes, being present of the lumen of the liposome, where the liposome may be combined with antibodies to surface membrane proteins or basement membrane proteins, ligands for cellular receptors, or other site directing compound, to localize the subject compounds to a particular target. (See, for example, Theresa and Mouse, Adv. Drug Delivery Rev. 1993, 21,117-133; Huwyler and Partridge, Proc. Natl. Acad. Sci. USA 1996, 93, 11421-11425; Dzau et al., Proc. Natl. Acad. Sci. USA 1996, 93,11421-11425; and Zhu et al., Science, 1993, 261, 209-211.) These compounds may be administered by catheter to localize the subject compounds to a particular organ or target site in the host. Generally, the concentration at the site of interest may be at least about 0.1 nM (e.g., intracellular or in the extracellular medium, preferably at least about 1 nM, usually not exceeding 1 mM, more usually not exceeding about 100 nM). To achieve the desired intracellular concentration, the concentration of the compound extracellularly will generally be greater than the desired intracellular concentration, ranging from about 2 to 1000 times or greater the desired intracellular concentration. Of course, where the toxicity profile allows for higher concentrations than those indicated for intracellular or extracellular concentrations, the higher concentrations may be employed, and similarly, where the affinities are high enough, and the effect can be achieved with lower concentrations, the lower concentrations may also be employed.

The subject compounds can be used to modulate physiological processes in vivo for a variety of reasons. In non-primates, particularly domestic animals, in animal husbandry and breeding, one can affect the development of the animal by controlling the expression of particular genes, modify physiological processes, such as accumulation of fat, growth, response to stimuli, etc. One can also use the subject compounds for therapeutic purposes in mammals. Domestic animals include feline, murine, canine, lagomorpha, bovine, ovine, canine, porcine, etc.

The subject compounds may used therapeutically to regulate (e.g., inhibit) proliferation of particular target cells in a mammalian host, regulate (e.g., inhibit) the expression of one or more genes related to an indication, change the phenotype of cells, either endogenous or exogenous to the host, where the native phenotype is detrimental to the host. Thus, by providing for binding to housekeeping or other genes of bacteria or other pathogen, particularly genes specific to the pathogen, one can provide for regulation (e.g., inhibition) of proliferation of the particular pathogen. Various techniques may be used to enhance transport across the bacteria wall, such as various carriers or sequences, such as polylysine, poly(E-K), nuclear localization signal, cholesterol and cholesterol derivatives, liposomes, protamine, lipid anchored polyethylene glycol, phosphatides, such as dioleoxyphosphatidylethanolamine, phosphatidyl choline, phosphatidylglycerol, α-tocopherol, cyclosporin, etc. In many cases, the subject compounds may be mixed with the carrier to form a dispersed composition and used as the dispersed composition. Similarly, where a gene may be essential to proliferation or protect a cell from apoptosis, where such cell has undesired proliferation, the subject compounds can be used to regulate (e.g., inhibit) the proliferation by regulating transcription of essential genes. This may find application in situations such as cancers, such as sarcomas, carcinomas and leukemias, restenosis, psoriasis, lymphopoiesis, atherosclerosis, pulmonary fibrosis, primary pulmonary hypertension, neurofibromatosis, acoustic neuroma, tuberous sclerosis, keloid, fibrocystic breast, polycystic ovary and kidney, scleroderma, rheumatoid arthritis, ankylosing spondilitis, myelodysplasia, cirrhosis, esophageal stricture, sclerosing cholangitis, retroperitoneal fibrosis, etc. Inhibition may be associated with one or more specific growth factors, such as the families of platelet-derived growth factors, epidermal growth factors, transforming growth factor, nerve growth factor, fibroblast growth factors (e.g., basic and acidic, keratinocyte fibroblast growth factor, tumor necrosis factors, interleukins, particularly interleukin 1, interferons, etc.). In other situations, one may wish to regulate (e.g., inhibit) a specific gene which is associated with a disease state, such as mutant receptors associated with cancer, inhibition of the arachidonic cascade, inhibition of expression of various oncogenes, including transcription factors, such as ras, myb, myc, sis, src, yes, fps/fes, erbA, erbB, ski, jun, crk, sea, rel, fms, abl, met, trk, mos, Rb-1, etc. Other conditions of interest for treatment with the subject compounds include inflammatory responses, skin graft rejection, allergic response, psychosis, sleep regulation, immune response, mucosal ulceration, withdrawal symptoms associated with termination of substance use, pathogenesis of liver injury, cardiovascular processes, neuronal processes, particularly where specific T-cell receptors are associated with autoimmune diseases, such as multiple sclerosis, diabetes, lupus erythematosus, myasthenia gravis, Hashimoto's disease, cytopenia, rheumatoid arthritis, etc., the expression of the undesired T-cell receptors may be diminished, so as to inhibit the activity of the T-cells. In cases of reperfusion injury or other inflammatory insult, one may provide for regulation (e.g., inhibition) of enzymes associated with the production of various factors associated with the inflammatory state and/or septic shock, such as TNF, enzymes which produce singlet oxygen, such as peroxidases and superoxide dismutase, proteases, such as elastase, INFγ, IL-2, factors which induce proliferation of mast cells, eosinophils, IgG₁, IgE, regulatory T cells, etc., or modulate expression of adhesion molecules in leukocytes and endothelial cells.

Other opportunities for use of the subject compounds include modulating levels of receptors, production of ligands, production of enzymes, production of factors, reducing specific cell populations, changing phenotype and genotype of cells, particularly as associated with particular organs and tissues, modifying the response of cells to drugs or other stimuli (e.g., enhancing or diminishing the response), inhibiting one of two or more alleles, repressing expression of target genes, particularly as related to clinical studies, modification of behavior, modification of susceptibility to disease, response to stimuli, response to pathogens, response to drugs, therapeutic or substances of abuse, etc.

Individual compounds may be employed, or alternatively combinations may be used which are directed to the same dsDNA region but different target sequences (e.g., contiguous or distal) or different DNA regions. Depending upon the number of genes which one wishes to target, a composition having one or a plurality of compounds or pairs of compounds which may be directed to different target sites may be used.

The subject compounds may be used as a sole therapeutic agent or in combination with other therapeutic agents. Depending upon the particular indication, other drugs may also be used, such as antibiotics, antisera, monoclonal antibodies, cytokines, anti-inflammatory drugs, and the like. The subject compounds may be used for acute situations or in chronic situations, where a particular regimen is devised for the treatment of the patient. The compounds may be prepared in physiologically acceptable media and stored under conditions appropriate for their stability. They may be prepared as powders, solutions or dispersions, in aqueous media, alcohols (e.g., ethanol and propylene glycol, in conjunction with various excipients, etc.). The particular formulation will depend upon the manner of administration, the desired concentration, ease of administration, storage stability, and the like. The concentration in the formulation will depend upon the number of doses to be administered, the activity of the compounds, the concentration needed as a therapeutic dosage, and the like. The subject compounds may be administered orally, parenterally (e.g., intravenously), subcutaneously, intraperitoneally, transdermally, etc. The subject compounds may be formulated in accordance with conventional ways, associated with the mode of treatment. As a result of the formulation, the subject compounds may be introduced into the cells, either as a directed introduction to a specific cell target or as random introduction into a number of different cell types. However, the subject compounds may only have an effect in those cells in which the target dsDNA is being transcribed or there is some other mechanism whereby the binding of the subject compounds can affect the mechanism. In this way selectivity can be achieved, since the only productive result will be in cells where the target dsDNA has an effect which is modified by the binding of the subject compounds to the dsDNA.

In view of the foregoing, it is to be noted that it has been discovered that the binding affinity and selectivity of, for example, a polyamide oligomer or polymer, to a target nucleotide sequence in the minor groove of dsDNA, may be altered by replacing one or more moieties therein which interact with a guanine nucleotide of the target sequence with a fused, bicyclic structure wherein at least one of the rings thereof is heteroaromatic (and more specifically the entire structure is heteroaromatic), the heteroatom therein acting as a hydrogen bond acceptor for interacting with the guanine nucleotide. Accordingly, it is to be understood that the compounds of the present invention (e.g., analogs of a polyamide oligomers or polymers, wherein one or more amido linkers or moieties are replaced by the insertion of fused, bicyclic structures as described herein) is widely applicable. For example, it may be utilized in a number of different applications, such as those described for known polyamides, it may essentially be prepared using known methods of polyamide preparation, it may be employed to bind dsDNA in the minor groove using methods, and in a manner, similar to those of known polyamides. (See, e.g., PCT Application Nos.: WO 98/35702; 98/37066; 98/37067; 98/37087; 98/45284; 98/49142; 98/5005; 98/50582; 00/15209; 00/15242; 00/15773; 00/04605; 01/48179; 02/04476; 02/34295; as well as, for example, U.S. Pat. Nos. 5,998,140; 6,143,901; 6,403,302; 6,472,537; and, 6,303,312; all of these are incorporated herein by reference for all relevant purposes.) For example, it may be utilized to prepare a cell which comprises a triplex of dsDNA and the compound of the present invention (see, e.g., PCT Application No. WO 98/50058 and U.S. Patent No. 5,998,140), it may be utilized to modulate expression (see, e.g., PCT Applications Nos. WO 98/35702 and 00/40605), and in various other methods of treatment (see, e.g., PCT Application Nos. WO 00/15209, 00/15242 and 00/15773). Additionally, it may be used, in some form or manner as described or illustrated herein or as known in the art for polyamides generally, to regulate replication by, for example, (1) interfering with the formation of the replication complex for bacteria and DNA viruses, or (2) assist in the action of natural defenses (including, for example, immune responses and enzyme reactions) against such pathogens by altering the structure, methylation patterns, or other properties of the viral or bacterial DNA.

The compounds of the invention may be utilized in one embodiment as a salt; that is, in one embodiment the present invention is directed to the compounds disclosed herein or a pharmaceutically acceptable salt thereof. The various salts of the present compound that may be employed generally include all those known to one of ordinary skill in the art, or which could be determined by one of ordinary skill in the art using known techniques.

Finally, the compounds of the present invention may be part of a diagnostic kit, wherein for example they is packaged in an appropriate container (e.g., vial, ampule, etc.), the kit further comprising for example external packaging (e.g., box or other container) to protect and support the storage container of the compound.

The following Examples are offered by way of illustration, and not by way of limitation.

### EXAMPLES

### EXAMPLE 1

Synthesis of 1-methyl-1H-benzoimidazole-5-carboxylic acid

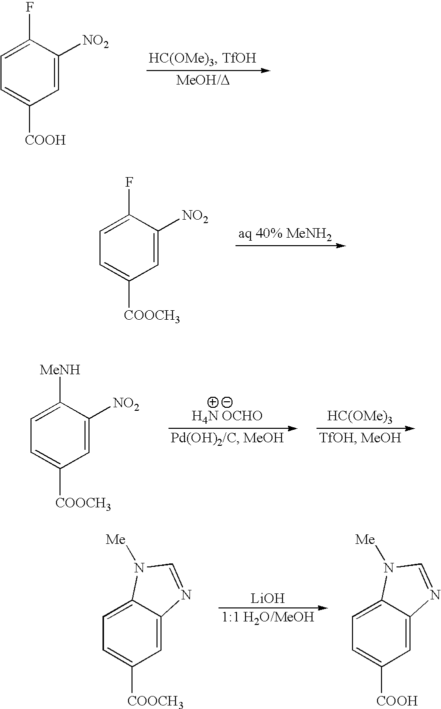

4-Fluoro-3-nitro-benzoic acid methyl ester: Trifluoromethanesulfonic acid (600 μL, 6.8 mmol) was added to a solution of 4-fluoro-3-nitro-benzoic acid (25.00 g, 135 mmol) and trimethyl orthoformate (29.5 mL, 270 mmol) in MeOH (250 mL), which was refluxed and monitored to completion over 4 days with 1H-NMR. The resulting solution was concentrated to remove most of the methanol, and then was diluted with 2 volumes of water to precipitate the product. The filter cake was washed with additional water and vacuum dried to afford 25.45 g (95%) desired product as a slightly off-white solid. ¹H NMR (300 MHz, CDCl₃) δ 8.68 (dd, J=2.2 Hz, J=7.25 Hz, 1H), 8.35 (ddd, J=2.2 Hz, 1H), 7.55 (dd, J=10.8 Hz, J=8.8 Hz, 1H), 3.96 (s, 3H). ¹⁹F NMR (300 MHz, CDCl₃) δ −114.33 (symmetric 7 line multiplet).

4-Methylamino-3-nitro-benzoic acid methyl ester. Aqueous 40% MeNH₂ (26.00 mL, 303 mmol) was added dropwise to an ice-water cooled solution of methyl 4-fluoro-3-nitro-benzoate (20.00 g, 101 mmol) in MeOH (100 mL) at a rate which maintained the internal reaction temperature<40° C. The resulting yellow slurry was stirred without external cooling for 15 min., during which the exotherm ceased. The solid was collected by filtration and washed with H₂O, then was vacuum dried to afford 20.79 g (99%) of product as a bright yellow solid. ¹H NMR (300 MHz, CD₃CN) δ 8.69 (d, J=2.0 Hz, 1H), 8.28 (br s, 1H), 8.02 (ddd, J=9.1 Hz, J=2.1 Hz, J=0.7 Hz, 1H), 6.99 (d, J=9.1 Hz, 1H), 3.85 (s, 3H), 3.04 (d, J=2.1 Hz, 3H).

1-Methyl-1H-benzoimidazole-5-carboxylic acid methyl ester. A slurry of Pearlman's catalyst (Pd(OH)₂ on carbon, 200 mg) in a small volume of methanol was added to a slurry of 4-methylamino-3-nitro-benzoic acid methyl ester (15.00 g, 71.40 mmol) and ammonium formate (22.50 g, 357 mmol) in MeOH (220 mL) at room temperature. Occasional cooling with a cool water bath controlled the resulting mild exotherm, and after 1 h the mixture had completely decolorized. This reaction mixture was filtered through a bed of celite and concentrated to remove most of the MeOH, then was partitioned between EtOAc and saturated aqueous NaHCO₃. The EtOAc phase was dried (MgSO₄) and concentrated to afford 12.92 g of crude methyl 4-methylamino-3-aminobenzoate as a brown solid. This compound was dissolved in MeOH (70 mL) with external warming, then trimethylorthoformate (15.60 mL, 143 mmol) and 1 drop of triflic acid were added and the mixture stirred overnight at RT. After concentration to remove most of the MeOH, the resulting solid was warmed to form an oil that was crystallized from ether. The solid product was collected by filtration and vacuum dried to give 10.70 g of desired product as a green solid. ¹H NMR (300 MHz, CD₃OD) δ 8.36 (s, 1H), 8.24 (s, 1H), 8.02 (dd, J=8.65 Hz, J=1.51 Hz, 1H), 7.62 (d, J=8.56 Hz, 1H), 4.85 (s, 3H), 3.93 (d, J=0.9 Hz, 3H).

1-Methyl-1H-benzoimidazole-5-carboxylic acid: H₂O (100 mL) was added to a solution of methyl 1-methyl-1H-benzoimidazole-5-carboxylate (10.00 g, 52.6 mmol) in 1N LiOMe in MeOH (100 mL, 100 mmol). The resulting solution was stirred under an inert atmosphere and monitored to completion by ¹H NMR. Aqueous 1N HCl (100 mL, 100 mmol) was added dropwise to the reaction mixture cooled with an ice-water bath. The gray solid which formed was collected by filtration and washed with water followed with methanol. Vacuum drying afforded 8.60 g of desired product. ¹H NMR (300 MHz, d₆-DMSO) δ 12.75 (br s, 1H), 8.31 (s, 1H), 8.23 (s, 1H), 7.89 (d, J=8.36 Hz, 1H), 7.62 (d, J=8.35 Hz, 1H), 3.85 (s, 3H).

### EXAMPLE 2

### Synthesis of 2-(4-tert-Butoxycarbonylamino-1-methyl-1H-pyrrol-2-yl)-1-methyl-1H-benzoimidazole-5-carboxylic acid

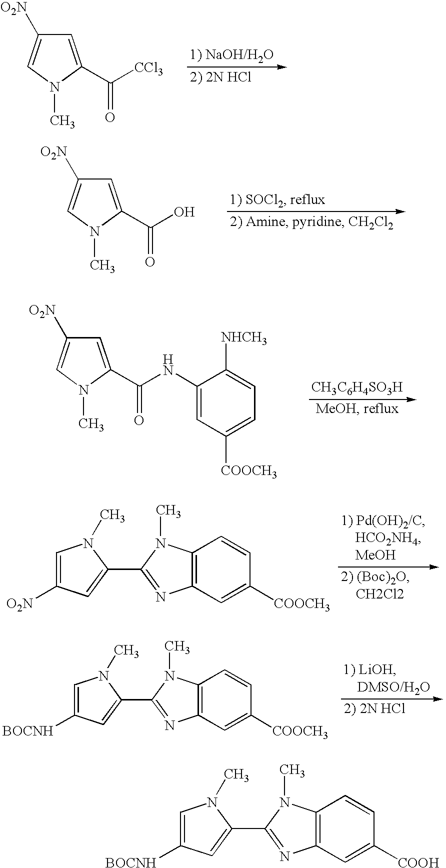

4-Nitro-1-methylpyrrole-2-carboxylic acid: A solution of NaOH (5.64 g, 140.9 mmol) in water (250 mL) was added 4-nitro-2-(trichloroacetyl)-1-methylpyrrole (12.75 g, 47.0 mmol) at room temperature. The mixture was stirred for 5 hours at room temperature, then was extracted with ethyl acetate (80 mL). The aqueous layer was acidified with 2N HCl to pH=3, and the resulting solid was filtered and dried in vacuo to afford 7.71 g (97%) of product as a white solid. ¹H NMR (300 MHz, CDCl₃) δ 7.65 (d, J=2.1 Hz, 1H), 7.55 (d, J=2.1 Hz, 1H), 4.01 (s, 3H). Anal. Calcd for C₆H₆N₂O₄: C, 42.36; H, 3.55; N, 16.47. Found: C, 42.53; H, 3.60; N, 16.49.

Methyl 3-((4-Nitro-1-methylpyrrole-2-yl)carbonyl)amino-4-methylamino-benzoate: A solution of 4-nitro-1-methylpyrrole-2-carboxylic acid (7.67 g, 45.1 mmol) in SOCl₂ (20 mL) was heated to reflux for 3 h. Excess SOCl₂ was then removed under vacuum, and the residue was dissolved in CH₂Cl₂ (150 mL) and added over 30 min to an ice-water cooled solution of methyl 3-amino-4-methylaminobenzoate (8.53 g, 47.3 mmol) and pyridine (7.30 mL, 90 mmol) in CH₂Cl₂ (650 mL). After stirring the reaction mixture for 2 days at room temperature, the solid was isolated by filtration, washed with CH₂Cl₂, and dried in vacuo to afford 12.04 g of product as a pale yellow solid. Concentration of the filtrate to 100 mL in vacuo resulted in crystallization of a second crop of 1.86 g of product. Total yield was 93%. ¹H NMR (300 MHz, d₆-DMSO) δ 9.56 (s, 1H), 8.18 (d, J=1.8 Hz, 1H), 7.72 (dd, J=8.7 Hz, J=1.8 Hz, 1H), 7.71 (d, J=1.8 Hz, 1H), 7.67 (d, J=1.8 Hz, 1H), 6.63 (d, J=8.7 Hz, 1H), 3.91 (s, 3H), 3.75 (s, 3H), 2.77 (s, 3H); ¹³C NMR (75 MHz, d₆-DMSO) δ 167.1, 160.3, 150.1, 134.8, 130.5, 129.9, 129.1, 127.3, 122.1, 116.2, 110.0, 109.7, 52.3, 38.4, 46.1. Anal. Calc'd. for C₁₅H₁₆N₄O₅: C, 54.21; H, 4.85; N, 16.86. Found: C, 54.39; H, 4.87; N, 16.82.

Methyl 2-(4-Nitro-1-methylpyrrole-2-yl)-1-methylbenzimidazole-5-carboxylate: A solution of methyl 3-((4-nitro-1-methylpyrrole-2-yl)carbonyl)amino-4-methylamino-benzoate (13.90 g, 41.8 mmol) and p-toluenesulfonic acid monohydrate (7.96 g, 41.8 mmol) in methanol (900 mL) was heated to reflux for 5 hours. The resulting mixture was poured into saturated aqueous Na₂CO₃ (500 mL), and then additional water (1000 mL) was added. The solid that formed was isolated by filtration and washed with water, and then methanol. Vacuum drying afforded 12.43 g (95%) of desired product as a white solid. ¹H NMR (300 MHz, CDCl₃) δ 8.53 (d, J=1.2 Hz, 1H), 8.11 (dd, J=8.7 Hz, J=1.5 Hz, 1H), 7.74 (d, J=1.5 Hz, 1H), 7.45 (d, J=8.7 Hz, 1H), 7.12 (d, J=1.8 Hz, 1H), 4.06 (s, 3H), 3.97 (s, 3H), 3.96 (s, 3H). Anal. Calc'd. for C₁₅H₁₄N₄O₄: C, 57.32; H, 4.49; N, 17.83. Found: C, 57.49; H, 4.53; N, 17.86.

Methyl 2-(4-tert-Butoxycarbonylamino-1-methylpyrrole-2-yl)-1-methylbenzimidazole-5-carboxylate: Under a nitrogen atmosphere, 20% Pd(OH)₂/C (1.05 g) was added to a solution of methyl 2-(4-nitro-1-methylpyrrole-2-yl)-1-methylbenzimidazole-5-carboxylate (12.33 g, 39.2 mmol) and ammonium formate (12.4 g, 196.0 mmol) in methanol (750 mL) at room temperature. To control the rate of gas evolution, the mixture was first heated to 50° C. for 1 h, and then at reflux for 1 h. The catalyst was removed by filtration of the reaction mixture through a pad of celite, and the filtrate was concentrated to remove most of the methanol, and then was diluted with CH₂Cl₂ (700 mL) and washed with aqueous 5% NaHCO₃ (200 mL) followed with saturated aqueous NaCl (200 mL). After drying over Na₂SO₄, the organic solution was reacted with di-tert-butyl dicarbonate (9.50 g, 43.5 mmol) overnight at room temperature. Concentration afforded a crude product which was purified by silica gel chromatography eluted with 1:1 hexane-ethyl acetate to afford 13.57 g (90%) of desired product as a white solid. ¹H NMR (400 MHz, CDCl₃) δ 8.48 (t, J=0.8 Hz, 1H), 8.01 (dd, J=8.8 Hz, J=1.2 Hz, 1H), 7.34 (d, J=8.4 Hz, 1H), 7.02 (s, 1H), 6.55 (s, 1H), 6.36 (s, 1H), 3.94 (s, 3H), 3.88 (s, 3H), 3.86 (s, 3H), 1.51 (s, 9H); ¹³C NMR (100 MHz, CDCl₃) δ 167.8, 148.4, 142.6, 139.1, 124.6, 124.3, 121.9, 116.8, 109.2, 105.1, 65.2, 52.2, 36.2, 32.0, 28.6. Anal. Calc'd. for C₂₀H₂₄N₄O₄: C, 62.49; H, 6.29; N, 14.57. Found: C, 62.56; H, 6.22; N, 14.57.

2-(4-tert-Butoxycarbonylamino-1-methylpyrrole-2-yl)-1-methylbenzimidazole-5-carboxylic acid: Lithium hydroxide monohydrate (7.36 g, 175.0 mmol) was added to a solution of methyl 2-(4-tert-butoxycarbonylamino-1-methylpyrrole-2-yl)-1-methylbenzimidazole-5-carboxylate (13.48 g, 35.0 mmol) in DMSO (200 mL) and water (50 mL). After 2 days at room temperature, the reaction was diluted with water (800 mL) and extracted with CH₂Cl₂ (150 mL) followed with ethyl acetate (150 mL). The aqueous solution was then acidified to pH 4 with 2N HCl to precipitate the carboxylic acid. After isolation by filtration and washing with water, the solid was recrystallized from methanol to afford 12.34 g (96%) of desired product as a white solid. ¹H NMR (400 MHz, d₆-DMSO) δ 12.65 (s, 1H), 9.06 (s, 1H), 8.15 (d, J=1.2 Hz, 1H), 7.83 (dd, J=8.8 Hz, J=1.6 Hz, 1H), 7.58 (d, J=8.4 Hz, 1H), 7.01 (s, 1H), 6.47 (s, 1H), 3.83 (s, 3H), 3.79 (s, 3H), 1.41 (s, 9H). Anal. Calc'd. for C₁₉H₂₂N₄O₄: C, 61.61; H, 5.99; N, 15.13. Found: C, 58.76; H, 6.51; N, 13.67.

### EXAMPLE 3

### Synthesis of 2-(2-tert-Butoxycarbonylamino-ethyl)-1-methyl-1H-benzoimidazole-5-carboxylic acid

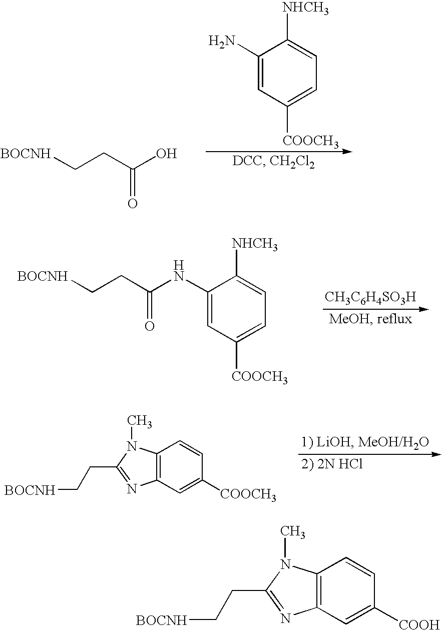

Methyl 3-((2-(tert-butoxycarbonylamino)ethyl)carbonyl)amino-4-methylamino-benzoate: DCC (12.3 g, 59.7 mmol) was added to an ice-water cooled solution of N—BOC β-alanine (10.0 g, 52.8 mmol) and methyl 3-amino-4-methylaminobenzoate (8.27 g, 45.9 mmol) in CH₂Cl₂ (400 mL). The reaction mixture was stirred 1 h at 0° C. and overnight at room temperature, then the insoluble urea was removed by filtration. After concentration, the residue was dissolved in ethyl acetate and washed with 5% NaHCO₃, followed with saturated NaCl. The organic solution was dried over Na₂SO₄, concentrated, and purified by chromatography over silica gel eluted with 1:2 hexane-EtOAc to afford 15.02 g (93%) of desired product as a white solid. ¹H NMR (300 MHz, d₆-DMSO) δ 9.06 (s, 1H), 7.70 (d, J=1.8 Hz, 1H), 7.66 (dd, J=8.4 Hz, J=1.8 Hz, 1H), 6.87 (s, 1H), 6.59 (d, J=8.4 Hz, 1H), 5.89 (d, J=4.8 Hz, 1H), 3.74 (s, 3H), 3.23 (q, J=6.9 Hz, 2H), 2.76 (d, J=4.8 Hz, 3H), 2.46 (t, J=6.9 Hz, 2H), 1.38 (s, 9H). Anal. Calc'd. for C₁₇H₂₅N₃O₅: C, 58.11; H, 7.17; N, 11.96. Found: C, 58.28; H, 7.09; N, 11.92.

Methyl 2-(2-(tert-butoxycarbonyl)amino)ethyl-1-methylbenzimidazole-5-carboxylate: A solution of methyl 3-((2-(tert-butoxycarbonylamino)ethyl)-carbonyl)-amino-4-methylaminobenzoate (14.86 g, 42.3 mmol) and p-toluenesulfonic acid monohydrate (8.04 g, 42.3 mmol) in methanol (250 mL) was heated to reflux for 5 h. The volume was reduced to ˜100 mL in vacuo, then poured into saturated Na₂CO₃ (50 mL), followed by the addition of water (800 mL). The solid was which formed was collected by filtration and dried in vacuo to afford 12.85 g (91%) of the expected benzimidazole as a white solid. ¹H NMR (300 MHz, CDCl₃) δ 8.42 (d, J=0.9 Hz, 1H), 8.00 (dd, J=8.4 Hz, J=1.5 Hz, 1H), 7.32 (d, J=8.4 Hz, 1H), 5.52 (s, 1H), 3.94 (s, 3H), 3.77 (s, 3H), 3.71 (q, J=6.3 Hz, 2H), 3.09 (t, J=6.3 Hz, 2H), 1.41 (s, 9H). Anal. Calc'd. for C₁₇H₂₃N₃O₄: C, 61.25; H, 6.95; N, 12.60. Found: C, 61.38; H, 7.00; N, 12.63.

2-(2-(tert-Butoxycarbonyl)amino)ethyl-1-methylbenzimidazole-5-carboxylic acid: A solution of lithium hydroxide monohydrate (8.0 g, 190.5 mmol) in water (20 mL) was added to a solution of methyl 2-(2-(tert-butoxycarbonyl)amino)ethyl-1-methylbenzimidazole-5-carboxylate (12.7 g, 38.1 mmol) in methanol (250 mL) at room temperature. The reaction mixture was stirred overnight at room temperature, then was concentrated and partitioned between water (250 mL) and ethyl acetate (150 mL). Acidification of the aqueous phase to pH 4 with 2N HCl resulted in formation of a solid which was collected and vacuum dried. Recrystallization of this material from ethyl acetate afforded 10.50 g (86%) of pure carboxylic acid as a white solid. ¹H NMR (300 MHz, CD₃COCD₃) δ 8.34 (d, J=1.2 Hz, 1H), 7.97 (dd, J=8.4 Hz, J=1.5 Hz, 1H), 7.54 (d, J=8.4 Hz, 1H), 6.30 (s, 1H), 3.89 (s, 3H), 3.63 (q, J=6.3 Hz, 2H), 3.15 (t, J=6.6 Hz, 2H), 1.38 (s, 9H). Anal. Calc'd. for C₁₇H₂₃N₃O₄: C, 60.17; H, 6.63; N, 13.16. Found: C, 60.21; H, 6.73; N, 13.08.

### EXAMPLE 4

### Synthesis of 1-methyl-1H-pyrrolo[3,2-b]pyridine-2-carboxylic acid

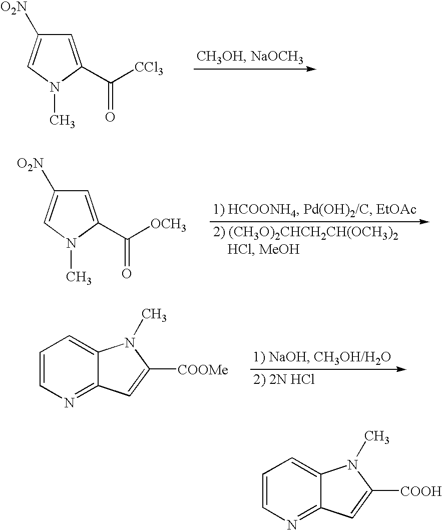

Methyl 1-methyl-4-nitropyrrole-2-carboxylate: To a solution of 4-nitro-2-(trichloroacetyl)-1-methylpyrrole (48.6 g, 179.0 mmol) in methanol (130 mL) was added NaOCH₃ (100 mg, 1.85 mmol) at room temperature. After exotherm ceased in 30 min, 98% H₂SO₄ (0.85 mL) and methanol (200 mL) were added. The mixture was heated to reflux until all the solid dissolved, then cooled to room temperature. The solid was collected by filtration and dried in vacuo to afford 30.34 g (92%) as a white solid. ¹H NMR (300 MHz, CDCl₃) δ 7.60 (d, J=1.8 Hz, 1H), 7.41 (d, J=1.8 Hz, 1H), 3.99 (s, 3H), 3.86 (s, 3H).

2-Methoxycarbonyl-1-methylpyrrolo(3,2-b)pyridine: A solution of methyl 1-methyl-4-nitropyrrole-2-carboxylate (10.02 g, 54.4 mmol) and HCO₂NH₄ (17.2 g, 272.0 mmol) in ethyl acetate (250 mL) was added 20% Pd(OH)₂/C. The mixture was heated to reflux for 2 h, then the catalyst was removed by filtration. The filtrate was evaporated in vacuo, then malonaldehyde bis(dimethyl acetal) (26.8 g, 163.2 mmol) and concentrated HCl (5 mL) were added, and the mixture was heated to reflux for 16 hours. After evaporation of the methanol in vacuo, a saturated aqueous solution of Na₂CO₃ (150 mL) was added, and the mixture was extracted with ethyl acetate (200 mL×2). The combined extracts were washed with saturated NaCl (150 mL), dried over Na₂SO₄, concentrated, and purified by chromatography with hexane-acetone (3:1) to afford 2.81 g (27.2%) of the desired product as a yellow solid. ¹H NMR (300 MHz, CDCl₃) δ 8.50 (dd, J=4.5 Hz, J=1.2 Hz, 1H), 7.61 (dd, J=5.5 Hz, J=1.2 Hz, 1H), 7.37 (s, 1H), 7.17 (dd, J=8.55 Hz, J=4.5 Hz, 1H), 4.00 (s, 3H), 3.89 (s, 3H); ¹³C NMR (75 MHz, CDCl₃) δ 162.2, 145.0, 143.4, 132.6, 130.0, 117.7, 110.1, 51.8, 31.5. Anal. Calc'd. for C₁₀H₁₀N₂O₂: C, 63.15; H, 5.30; N, 14.73. Found: C, 63.21; H, 5.30; N, 14.63.

1-Methyl-pyrrolo(3,2-b)pyridine-2-carboxylic acid: To a solution of 2-methoxycarbonyl-1-methylpyrrolo(3,2-b)pyridine (2.03 g, 10.7 mmol) in methanol (25 mL) and water (5 mL) was added NaOH (470 mg, 11.7 mmol) at room temperature. The mixture was stirred overnight at room temperature. After evaporation of the methanol in vacuo, water (15 mL) was added, then extracted with ethyl ether (50 mL). The aqueous extract was acidified with 2N HCl to pH=6 and stirred in an ice bath for 1 h. The solid was collected by filtration and dried in vacuo to afford 1.71 g (91%) of desired carboxylic acid as a pale yellow solid. ¹H NMR (300 MHz, CD₃OD) δ 8.46 (d, J=4.8 Hz, 1H), 8.18 (d, J=8.4 Hz, 1H), 7.44 (dd, J=8.7 Hz, J=4.8 Hz, 1H), 7.26 (s, 1H), 4.15 (s, 3H); ¹³C NMR (75 MHz, d₆-DMSO) δ 163.5, 145.3, 143.6, 133.1, 131.9, 119.8, 119.5, 109.5, 32.3. Anal. Calcd for C₉H₈N₂O₂: C, 61.36; H, 4.58; N, 15.9. Found: C, 60.84; H, 4.78; N, 15.44.

### EXAMPLE 5

### Synthesis of 2-(4-tert-butoxycarbonylamino-1-methyl-1H-pyrrol-2-yl)-benzothiazole-5-carboxylic acid

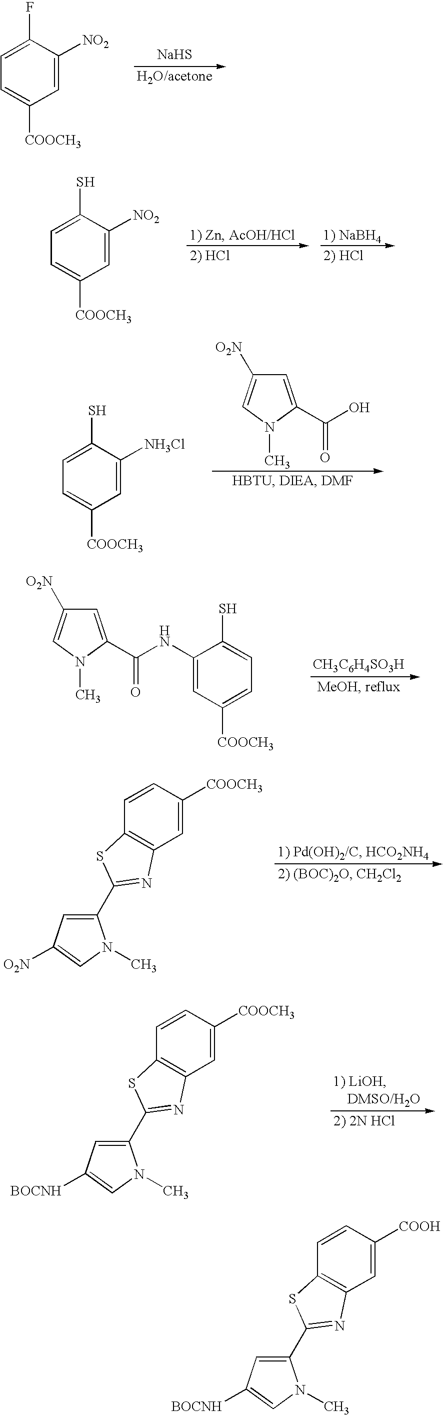

4-Mercapto-3-nitro-benzoic acid methyl ester A solution of methyl 4-fluoro-3-nitrobenzoate (5.00 g, 25.1 mmol) in acetone (25 mL) was added dropwise to an ice-water cooled solution of NaHS(xH₂O) (7.50 g, <134 mmol) in H₂O (25 mL). ¹H-NMR indicated complete reaction within 5 minutes, so concentrated HCl (5.00 mL) was added to quench the reaction. The precipitate that formed was collected by filtration, washed with water, and vacuum dried to afford 6.30 g of product as a pale yellow solid. ¹H NMR (400 MHz, CDCl₃) δ 8.88 (d, J=1.7 Hz, 1H), 8.04 (dd, J=8.3 Hz, J=1.9 Hz, 1H), 7.50 (d, J=8.3 Hz, 1H), 4.20 (s, 1H), 3.95 (s, 3H).

3-Amino-4-mercapto-benzoic acid methyl ester hydrochloride: Zn dust (22.08 g, 338 mmol) was added in portions over 30 minutes to an ice-water cooled suspension of methyl 4-mercapto-3-nitrobenzoate (6.00 g, 28.1 mmol) in a mixture of acetic acid (80 mL) and concentrated aqueous 37% HCl (42 mL). The resulting slurry was refluxed for 1 hour or until the reaction turned colorless, then was cooled to room temperature and treated with a solution of sodium acetate (32.00 g, 390 mmol) in water (320 mL). The solid was collected and vacuum dried, then was dissolved in concentrated aqueous 37% HCl (80 mL). After briefly warming for 10 minutes, the product was allowed to crystallize overnight in the freezer. This was collected by filtration and vacuum dried to afford 3 g of product as a pale yellow solid. Varying amounts of the symmetrical disulfide were formed in this reaction, so complete conversion to the mercaptan was achieved by reduction with a 4 fold excess of NaBH₄ in MeOH at ice-water temperature. After 30 minutes, the reaction was concentrated, diluted with water and acidified with aqueous HCl. The resulting mercaptan was collected by filtration and vacuum dried. ¹H NMR (400 MHz, d₆-DMSO) δ 7.36 (s, 2H), 7.18 (d, J=8.1 Hz, 2H), 7.00 (d, J=8.1 Hz, 2H), 3.75 (s, 6H). Positive electrospray LCMS m/e 184 (M+H⁺).

2-(1-Methyl-4-nitro-1H-pyrrol-2-yl)-benzothiazole-5-carboxylic acid methyl ester. 1-methyl-4-nitro-1H-pyrrole-2-carboxylic acid (2.00 g, 11.9 mmol) was activated with HBTU (5.50 g, 14.5 mmol) in DMF (50 mL), and monitored to completion by HPLC analysis. After 30 minutes, a solution of methyl 3-amino-4-mercapto-benzoate hydrochloride (2.60 g, 11.8 mmol) and DIEA (18.60 g, 144 mmol) in DMF (50 mL) was added and the resulting mixture was stirred at RT overnight. The DMF was removed under vacuum and the residue partitioned between EtOAc and diluted aqueous HCl. The EtOAc layer was washed with water, dried (MgSO₄), and concentrated to afford crude amide. A solution of this material was cyclized with 1 equivalent of TsOH monohydrate in refluxing methanol over 6 hours. After cooling to RT, the solid that formed was collected by filtration and vacuum dried. ¹H NMR (400 MHz, d₆-DMSO) δ 8.45 (d, J=1.2 Hz, 1H), 8.32 (d, J=1.6 Hz, 1H), 8.24 (d, J=8.5 Hz, 1H), 7.96 (dd, J=8.5 Hz, J=1.6 Hz, 1H), 7.49 (d, J=2.0 Hz, 1H), 4.10 (s, 3H), 3.86 (s, 3H). Positive electrospray LCMS m/e 318 (M+H⁺).

2-(4-tert-Butoxycarbonylamino-1-methyl-1H-pyrrol-2-yl)-benzothiazole-5-carboxylic acid: The methyl 2-(1-methyl-4-nitro-1H-pyrrol-2-yl)-benzothiazole-5-carboxylate from the above reaction was suspended in 1:1 MeOH/EtOAc (100 mL) and hydrogenated overnight at 60 psi H₂ over Pd/C. HPLC showed complete reaction, so 2 equivalents of (BOC)₂O were added to the reaction mixture. After stirring overnight at RT, HPLC again showed complete reaction with clean formation of the BOC protected amine: positive electrospray LCMS m/e 388 (M+H⁺). This mixture was filtered through a celite pad to remove the catalyst, then was concentrated and diluted with 4:1 MeOH/H₂O and reacted with excess 2N NaOH overnight at RT. The reaction mixture was partitioned between EtOAc and dilute aqueous HCl. The EtOAc layer was dried (MgSO₄) and concentrated to afford 1.60 g of desired carboxylic acid as a brown solid. ¹H NMR (400 MHz, d₆-DMSO) δ 12.85 (br s, 1H), 9.15 (s, 1H), 8.32 (d, J=1.5 Hz, 1H), 8.08 (d, J=8.3 Hz, 1H), 7.85 (dd, J=1.6 Hz, J=8.3 Hz, 1H), 7.08 (s, 1H), 6.66 (s, 1H), 3.97 (s, 3H), 1.41 (s, 9H). Positive electrospray LCMS m/e 374 (M+H⁺).

### EXAMPLE 6

### Solid Phase Synthesis of Polyamide or Polyamide Analogs

Solid phase synthesis of polyamide or polyamide analogs was performed using standard BOC protocol on an ABI 433A peptide synthesizer with 1 gm of BOC-β-alanine-Pam Resin (0.26 mmol/g) and four equivalents (1.0 mmol) of each subunit per synthesis cycle. The subunits were selected from the products of Examples 1-5 and included N-t-BOC-α-alanine, N-t-BOC-γ-aminobutyric acid, 1-methyl-4-(tert-butyloxycarbonylamino)-pyrrole-2-carboxylic acid, 1-methyl-4-(tert-butyloxycarbonylamino)imidazole-2-carboxylic acid, 1-methyl imidazole-2-carboxylic acid, and 1,3-benzothiazole-5- carboxylic acid.

Subunits (1.0 mmol) to be serially linked to the resin were weighed into individual synthesis cartridges. Each subunit was dissolved in 3 mL DMF and 2 mL DIEA just prior to its attachment to the resin. After mixing for 3 min the solution was transferred to the activator vessel and reacted with HBTU (1 mmol) in DMF (2 mL) for 10 min. Each cycle of subunit addition to the resin involved a series of repeating steps. Initially, deprotection of BOC-β-alanine-Pam Resin (1.0 g, 0.26 mmol) was carried out in a 41 mL vessel by reaction with 25% TFA/CH₂Cl₂ for 3 min, filtration, reaction with 50% TFA/CH₂Cl₂ for 16 min, and then washing with dichloromethane (4×7 mL). The deprotected β-alanine-Pam Resin was neutralized with a 10% solution of DIEA in dichloromethane followed with 10% DIEA in DMF, and then washed with DMF (6×5 mL). After deprotection and neutralization of the resin, a solution of activated subunit from the activator vessel was transferred to the reaction vessel and allowed to couple for 30 min. A solution of DMSO/NMP was then added and coupling was continued for another 45 min (total coupling time 75 min). Finally, DIEA (1 mL) was added and coupling continued for another 60 min. The reaction vessel was drained and the resin was washed with DMF, then capped with 10% acetic anhydride and 5% DIEA in DMF for 9 min. A final wash with dichloromethane and filtration completed the synthesis cycle. Additional synthesis cycles of BOC deprotection, subunit activation, and coupling were carried out to attach each subunit to the growing polyamide or polyamide analog on the resin. Cleavage of the polyamide or polyamide analog from the resin was achieved with 3-(dimethylamino)propylamine (6 mL) at 45° C. for 18 h. The resin was removed by filtration and washed with 12 mL of water. After HPLC analysis, the filtrate was evaporated on the rotary evaporator to remove water and 3-(dimethylamino)-propylamine. The residue was dissolved in 1:1 DMF/water (10-15 mL), filtered, and purified by preparative C₁₈ HPLC eluted with a linear gradient of 20-80% methanol in water containing 0.1% TFA. The fractions were analyzed by analytical HPLC, and the pure fractions were combined, concentrated, and lyophilized from a 25-50% mixture of tert-butanol and to afford each of the following polyamide or polyamide analogs as fluffy powders, which were characterized by high-resolution mass spectrometry.

The results of the solid phase syntheses are presented below in Table 1.

TABLE 1

| Identifier | Structure |
|---|---|
| IP₂IGP₄BDa | 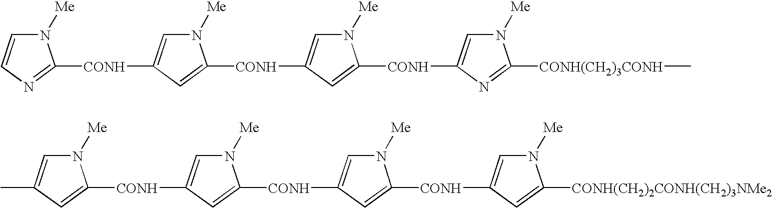 |
| BiP₂IGP₄BDa | 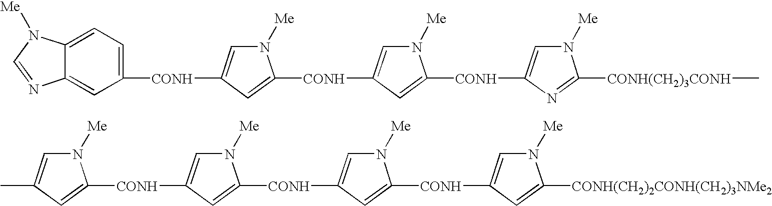 |
| IP₂BiGP₄BDa | 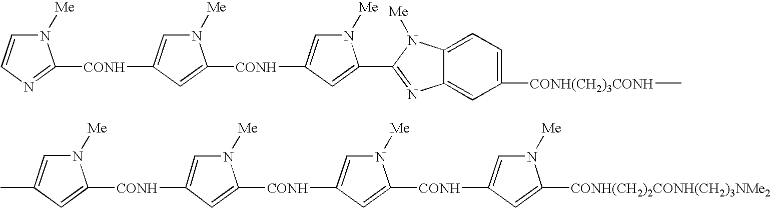 |
| BiP₂BiGP₄BDa | 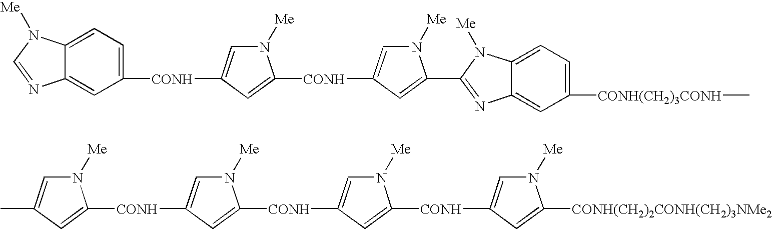 |
| BiPBBiGP₄BDa | 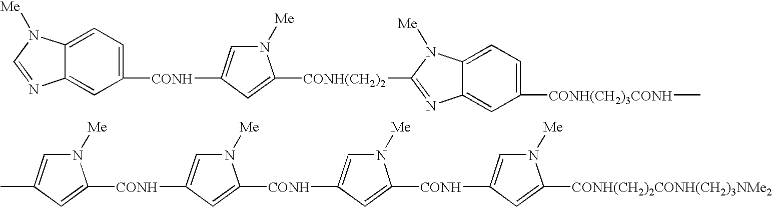 |
| PpP₂IGP₄BDa | 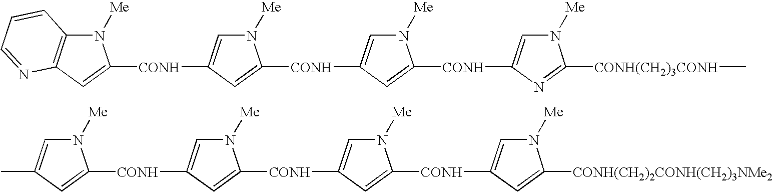 |
| BtP₂IGP₄BDa | 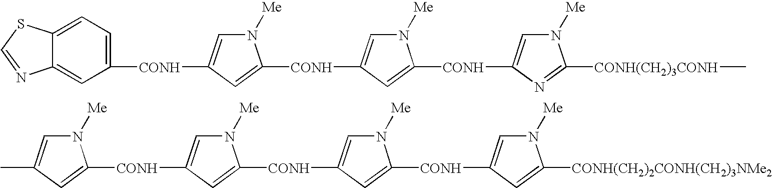 |
| BiP₂BtGP₄BDa | 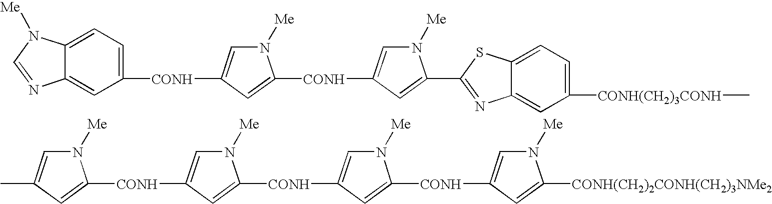 |
| BtP₂BtGP₄BDa | 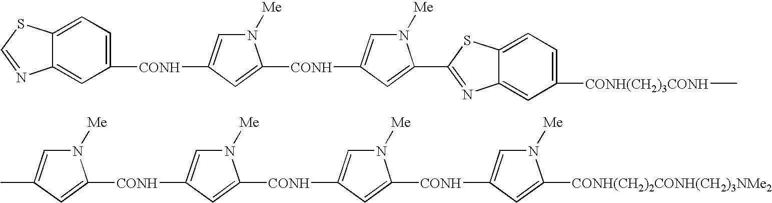 |

### EXAMPLE 7

### In Vitro Transcription-Translation Assay

Transcription-translation reactions were performed using S30 E. coli extract, plasmid DNA containing the lacZ promoter driving the β-gaiactosidase gene, and the FluoroTect™ Green_{lys} in vitro labeling system for protein detection. All of the above were purchased from Promega. The amount of plasmid DNA typically used was 0.5 μg. For transcription-translation reactions (assay volume 12.5 μL), typically, a master mix containing all reaction components except the polyamide or polyamide analog was prepared and kept on ice. For example, preparation of 20 reactions would require a master mix containing: 20 μL of plasmid DNA (stock concentration 500 μg/mL), 5 μL of complete amino acid mix, 100 μL of S30 premix, 75 μL of S30 extract, and 5 μL of tRNAlys-Bodipy. This mixture would be gently mixed and aliquoted into 20 tubes at 11.5 μL per tube followed by addition of 1 μL of a polyamide or polyamide analog of Example 6 or water. The reactions were then incubated at 30° C. for one hour followed by placement on ice for 5 min. to stop the reaction. A 5 μL aliquot of the reaction mixture was added to 20 μL of gel loading buffer (95% Laemlli buffer, 5% beta-mercaptoethanol) and heated to 65-70° C. for 10 min. The tubes were briefly centrifuged and 15 μL of each mixture was loaded onto 4-15% polyacrylamide gels (Criterion) purchased from BiORad. Gels were run using 1× Tris/SDS/glycine running buffer at 30 mA (constant current) and 120V max for 2 hrs. The gels were briefly rinsed using Millipore water and imaged using Molecular Dynamics Typhoon (Ex: 532 nm; Em: 526 nm). The protein bands in the gel were quantified using Molecular Dynamics ImageQuant software.

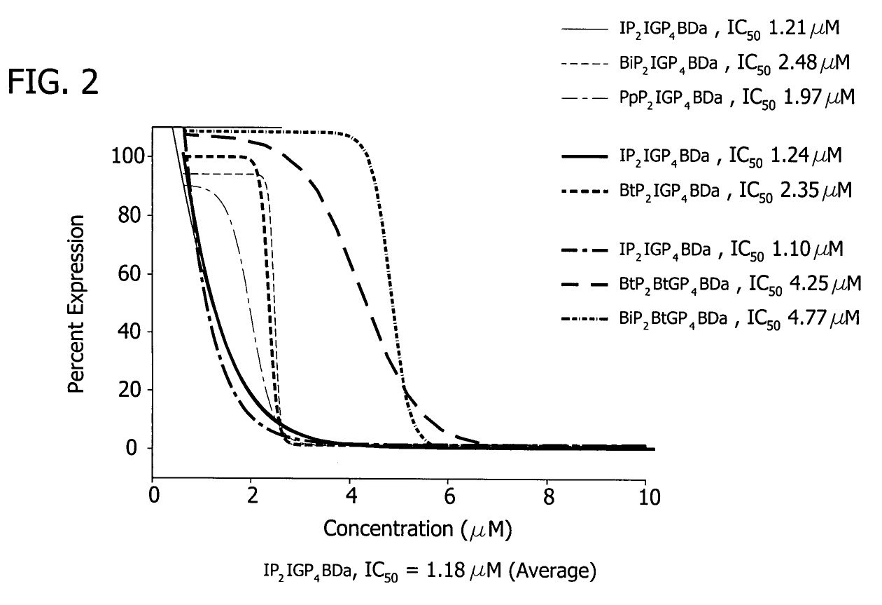

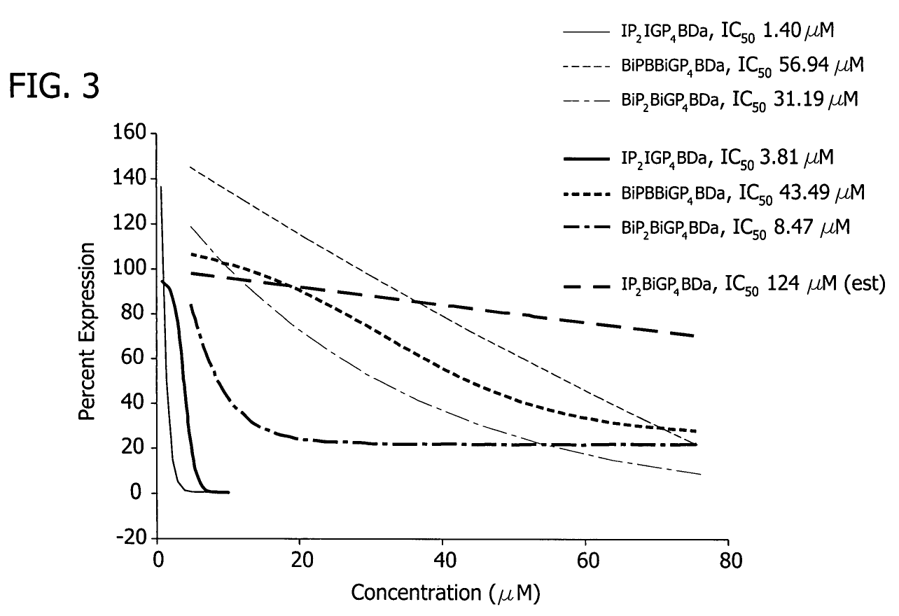

Plots or graphs of the data shown in the graphs of FIGS. 2 and 3 were used to determine the IC50 values for the noted polyamide or polyamide analogs of Example 6.

### EXAMPLE 8

### Polyamide or Polyamide Analog/DNA Binding Interactions Using Surface Plasmon Resonance (SPR)

A SA sensor chip coated with streptavidin (purchased from the BIAcore, Inc.) was employed for capturing 5′-BIOTIN-CGTATGTTGTGTGTTTTCACACA-ACATACG with a desirable density (150 RU or less) for binding studies. Polyamide or polyamide analog stock solutions (500 uM in DMSO) were prepared for each member in Table 1 of Example 6, and each was diluted with HBS-EP (10 mM Hepes, 150 mM NaCl, 3 mM EDTA, 0.005% p-20, pH 7.4, purchased from the BIAcore, Inc.) containing 0.1% DMSO to form a series of solutions (0 nM, 1.95 nM, 3.9 nM, 7.8 nM, 15.6 nM, 31.3 nM, 62.5 nM, 125 nM, 250 nM, 500 nM). The running buffer was HBS-EP containing 0.1% DMSO and the flow rate was set at 30 μL/min. In each binding experiment, a polyamide or polyamide analog sample was injected over the DNA surface for 4 minutes followed by 5 minutes of dissociation in the running buffer. The sensor chip surface was washed twice, one minute each time, with the running buffer, followed by a 40 second regeneration of the surface with 10 mM glycine, pH2.0 buffer for additional binding experiments.

The binding properties (on-rate, off-rate, binding affinity, and binding stoichiometry) were determined via a global fitting of the binding curves using the programs supplied with the BIAcore (Uppsala, Sweden) system. This program fits the entire association and dissociation data for all concentrations simultaneously to yield on-rate values (kₐ) and off-rate values (k_{d}). The affinity (K_{D}) was obtained from dividing the off-rate by the on-rate. The following Table (Table 2) lists the experimental results obtained for each polyamide or polyamide analog of Example 6 that was studied.

TABLE 2

| | | | Kinetic | Steady State |
|---|---|---|---|---|
| Identifier | kₐ (M⁻¹s⁻¹) | K_{d} (s⁻¹) | K_{D} (nM) | K_{D} (nM) |
| iP₂iGP₄BDa | 6.18 × 10⁶ | 8.00 × 10⁻⁴ | 0.129 | 0.486 |
| BiP₂IGP₄BDa | 2.18 × 10⁶ | 5.40 × 10⁻³ | 2.48 | 14.6 |
| IP₂BiGP₄BDa | | | >500 | |
| BiP₂BiGP₄BDa | | | >500 | |
| BiPBBiGP₄BDa | | | >500 | |
| PpP₂IGP₄BDa | 5.37 × 10⁶ | 3.48 × 10⁻² | 6.47 | 18.0 |
| BtP₂IGP₄BDa | 4.68 × 10⁶ | 1.76 × 10⁻² | 3.77 | 13.0 |
| BiP₂BtGP₄BDa | 2.82 × 10⁶ | 3.36 × 10⁻² | 11.9 | 13.6 |
| BtP₂BtGP₄BDa | 1.49 × 10⁶ | 3.95 × 10⁻² | 26.5 | 34.8 |

The on-rate (>1 E+6) of polyamide or polyamide analog binding to DNA is relatively fast when compared with those of antigen-antibody or ligand-receptor interaction (<1 E+6 in general). The fast on-rate of polyamide- or polyamide analog-DNA interaction required use of low density of DNA on the sensor chip surface to minimize the mass transport limit of the interaction. In these studies, biotinylated DNA was captured onto a streptavidin surface with a density of 150RU or lower. The binding properties were determined using a 1:1 reaction model with mass transfer limit and bulk shift variation in a global analysis. A steady state analysis was also made for the binding affinity. The binding affinity results obtained from the steady state analysis deviate somewhat from that of the kinetics analysis due to the fact that the variation of bulk shift of each sensorgram was not taken into consideration during the steady state analysis. This can be seen in a higher Rmax from the steady state analysis than in the kinetic analysis.

The binding affinities of IP₂BiGP₄BDa, BiP₂BiGP₄BDa, and BiPBBiGP₄BDa were estimated by injecting 500 nM of these compounds over the same DNA surface used for the study of other compounds. The amount bound of these compounds was determined and compared with that of the reference polyamide IP₂IGP₄BDa. These three compounds all bound much less than 50% of the reference indicating that the binding affinity is less than 500 nM. The binding properties of these three compounds were therefore not investigated further.

In a separate experiment the binding stoichiometry of reference polyamide IP₂IGP₄BDa to DNA was determined by saturating the low density of DNA surface (<150RU) with high concentration of IP₂IGP₄BDa (125 nM, 250 nM and 500 nM). The amount bound was determined and found to be similar to that of Rmax determined in a kinetics analysis as well as to the theoretical value for a 1:1 binding. The binding stoichiometry of the other five polyamide analogs was therefore calculated to be close to 1:1 using a similar approach. It was observed that BtP₂IGP₄BDa deviates significantly from that of a 1:1 binding stoichiometry.

In view of the foregoing, a strong correlation exists between the K_{D} values (Example 8) and IC₅₀ values (Example 7).

The results of Examples 6 and 7 show that the two imidazole units in IP₂IGP₄BDa may be replaced with heterocycles designed to alter the spacing between adjacent H-bond acceptor and H-bond donor moieties. Replacement of only the terminal imidazole unit with a benzimidazole produced BiP₂IGP₄BDa, which provided good inhibition (IC₅₀=2.48 μM) of the in vitro transcription/translation assay. Replacement of only the internal imidazole unit with a benzimidazole produced IP₂BiGP₄BDa, which provided less inhibition (IC₅₀>75 μM) of the in vitro transcription/translation assay (as compared to IP₂IGP₄BDa). Replacement of both imidazoles with benzimidazole produced BiP₂BiGP₄BDa, which provides a level of inhibition (IC₅₀=19.83 μM) that falls between BiP₂IGP₄BDa and IP₂BiGP₄BDa. Thus, replacement of IP₂IGP₄BDa internal imidazole is best tolerated when the terminal imidazole is also replaced, and results in uniform spacing between the adjacent H-bond acceptor and H-bond donor moieties that bind with the DNA minor groove.

The comparatively lesser inhibition achieved with IP₂BiGP₄BD may be due to a combination of unfavorable steric interactions between benzimidazole ring hydrogen atoms with the DNA minor groove, and/or between the benzimidazole N-methyl group with the adjacent pyrrole N-methyl group. The binding between the terminal imidazole H-bond accepting nitrogen with the G-NH₂ group in the minor groove may cause distorted geometries for the remaining polyamide or polyamide analog/DNA interactions due to nonuniform spacing between the adjacent H-bond acceptor and H-bond donor moieties that bind with the regularly spaced nucleotides along the DNA minor groove. This nonuniform spacing leads to an unfavorable steric interaction between benzimidazole ring hydrogen atoms and its complementary G-NH₂ group in the DNA minor groove. In BiP₂BiGP₄BDa, these unfavorable interactions are decreased because of the uniform spacing between the adjacent H-bond acceptor and H-bond donor moieties that bind with the DNA minor groove. The benzimidazole units each have an H-bond accepting nitrogen that can bind to the complementary G-NH₂ group which extends into the DNA minor groove. Although there is no (or much less) unfavorable steric interaction between the benzimidazole ring hydrogen atoms and the G-NH₂ group, the unfavorable steric interaction still exists between the benzimidazole N-methyl group and the adjacent pyrrole N-methyl group.

The steric interaction between the benzimidazole N-methyl group and the adjacent pyrrole N-methyl group was addressed through replacement of the internal imidazole unit with a benzothiazole unit, and replacement of the terminal imidazole unit with a benzimidazole, benzothiazole, or pyrrolopyridine unit. The resulting polyamide analogs (i.e., BiP₂BtGP₄BDa, BtP₂BtGP₄BDa, and PPp₂BtGP₄BDa) provided excellent inhibition of the in vitro transcription/translation assay, and were 2-fold less active than IP₂IGP₄BDa. The polyamide or polyamide analog/DNA binding results of Example 8 correlated with the in vitro transcription/translation assay inhibition data of Example 7, providing additional support for these binding interactions. Other ring systems are expected to provide greater binding affinity.

1. A synthetic and/or non-naturally occurring compound which binds a sequence of nucleotides with specificity in a minor groove of double-stranded DNA, said sequence containing at least one guanine nucleotide, the compound comprising at least one H-bond donor moiety and at least one H-bond acceptor moiety spaced apart to bind with specificity said sequence, wherein said H-bond acceptor moiety has a fused, bicyclic structure and is heteroaromatic, wherein said structure has a heteroatom therein which acts as a hydrogen bond acceptor to bind guanine in the minor groove of the dsDNA sequence, and wherein said structure cannot form a tautomer in which said heteroatom becomes a H-bond donor.

2. The compound of claim 1 wherein said fused, bicyclic structure occupies a first terminal position within the compound.

3. The compound of claim 1 wherein said compound comprises more than one of said fused, bicyclic structures, said structures being substantially the same.

4. The compound of claim 3 wherein the second ring of said terminal fused, bicyclic structure is directly bound via a carbon-carbon bond to a first ring of a second fused, bicyclic structure which occupies a non-terminal position within the compound.

5. The compound of claim 3 wherein the second ring of said terminal fused, bicyclic structure is indirectly bound to a first ring of a second fused, bicyclic structure via a linker which is a H-bond donor, said second fused, bicyclic structure occupying a non-terminal position within the compound.

6. The compound of claim 1 wherein said compound comprises more than one of said fused, bicyclic structures, said structures being different.

7. The compound of claim 6 wherein the second ring of said terminal fused, bicyclic structure is directly bound via a carbon-carbon bond to a first ring of a second fused, bicyclic structure which occupies a non-terminal position within the compound.

8. The compound of claim 6 wherein the second ring of said terminal fused, bicyclic structure is indirectly bound to a first ring of a second fused, bicyclic structure via a linker which is a H-bond donor, said second fused, bicyclic structure occupying a non-terminal position within the compound.

9. The compound of claim 1 wherein the second ring of said terminal fused, bicyclic structure is indirectly bound to another heteroaromatic moiety of the compound via a linker which is a H-bond donor.

10. The compound of claim 9 wherein said linker comprises a —NH— moiety which is the H-bond donor.

11. The compound of claim 1 wherein said fused, bicyclic structure comprises a 5-member and a 6-member ring.

12. The compound of claim 1 wherein said fused, bicyclic structure has two unsaturated rings and has a formula: 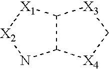
wherein:
X₁ and X₂ are independently selected from O, S, N, NR², CR³, CR⁴═CR⁴′, CR⁴═N, N═CR⁴, N═N and CR⁴″, provided that (i) when each one of X₁ or X₂ is independently selected from O, S or NR², the other is independently selected from CR³ or N, and (ii) when each one of X₁ or X₂ is independently selected from CR⁴═CR⁴′, CR⁴═N, N═CR⁴ or N═N, the other is independently selected from CR⁴″ or N;
X₃ is independently selected from N, O, S, CR⁵, NR⁵, CR⁵═CR⁵′, CR⁵═N, N═CR⁵ and N═N, and X₄ is independently selected from O, S, N and CH; provided that (i) when each X₃ is independently selected from CR⁵ or N, X₄ is independently selected from O or S, and (ii) when each X₃ is independently selected from O, S, NR⁵, CR⁵═CR⁵′, CR⁵═N, N═CR⁵ or N═N, X₄ is independently selected from CH or N; and further provided that (a) when said fused, bicyclic structure occupies a first terminal position within the compound, the carbon present between X₃ and X₄ is a point of attachment to the remaining portion of the compound; (b) when said fused, bicyclic structure occupies a non-terminal position within the compound, X₂ is a carbon atom which directly or indirectly serves as a point of attachment to the compound for the first ring of the structure, while the carbon atom between X₃ and X₄ serves as the point of attachment for the second ring thereof; and, (c) when more than one of said fused, bicyclic structures is present in the compound, said structures may be substantially the same or different; and,
each substituent R², R³, R⁴, R⁴¹, R⁴″, R⁵, R⁵′ is independently selected from H, hydroxy, N-acetyl, benzyl, substituted or unsubstituted C₁₋₆ alkyl, substituted or unsubstituted C₁₋₆ alkylamine, substituted or unsubstituted C₁₋₆ alkyldiamine, substituted or unsubstituted C₁₋₆ alkylcarboxylate, substituted or unsubstituted C₂₋₆ alkenyl, substituted or unsubstituted C₂₋₆ alkynyl and, when attached to a carbon atom, optionally halo, provided that (i) when X¹ or X² is NR², R² is other than H, and (ii) when X³ is NR⁵, R⁵ is other than H.

13. The compound of claim 12 wherein said compound comprises at least about 2 non-fused, non-bicyclic heteroaromatic moieties, which may be substituted or unsubstituted and which may be the same or different.

14. The compound of claim 13 wherein said non-fused, non-bicyclic heteroaromatic moieties are selected from substituted or unsubstituted pyrrole, substituted or unsubstituted furan, substituted or unsubstituted thiophene, substituted or unsubstituted pyrazole, substituted or unsubstituted isothiazole, substituted or unsubstituted isoxazole, or a combination thereof.

15. The compound of claim 13 wherein said non-fused, non-bicyclic heteroaromatic moieties are oriented such that a heteroatom therein is not directed toward the floor of the minor groove of said dsDNA.

16. The compound of claim 15 wherein said non-fused, non-bicyclic heteroaromatic moieties are selected from substituted or unsubstituted oxazole, substituted or unsubstituted thiazole, substituted or unsubstituted imidazole, substituted or unsubstituted triazole, substituted or unsubstituted oxadiazole, substituted or unsubstituted thiadiazole, or a combination thereof.

17. The compound of claim 13 wherein said non-fused, non-bicyclic heteroaromatic moieties contain one or more nitrogen heteroatoms.

18. The compound of claim 17 wherein said heteroaromatic moieties are substituted, said moieties being independently selected from N-hydroxy, N-acetyl, N-benzyl, N—C₁₋₆ alkyl, N—C₁₋₆ alkylamine, N—C₁₋₆ alkyldiamine, N—C₁₋₆ alkylcarboxylate, N—C₂₋₆ alkenyl and N—C₂₋₆ alkynyl.

19. The compound of claim 18 wherein one or more of said heteroaromatic moieties are pyrrole.

20. The compound of claim 19 wherein one or more of said moieties are N-methylpyrrole.

21. The compound of claim 13 wherein said compound further comprises at least one aliphatic amino acid moiety.

22. The compound of claim 21 wherein said aliphatic amino acid is chosen from the group consisting of glycine, β-alanine, γ-aminobutyric acid, 5-aminovaleric acid, 2-methoxy-α-alanine and 2,4-diaminobutyric acid.

23. The compound of claim 22 wherein said aliphatic amino acid forms a hairpin linkage between said heteroaromatic moieties.

24. The compound of claim 1 wherein said compound has the structure: 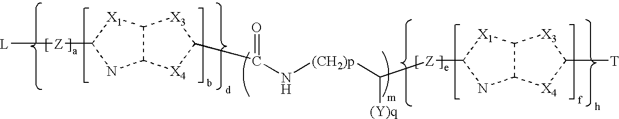
wherein:
L is independently selected from (i) H, (ii) H₂N(HN)CNHCH₂, the terminal methylene group, CH₂, being attached to the carbonyl carbon, and (iii) a non-tautomerizing, fused bicyclic structure: 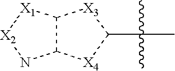
and further wherein each ring of each non-tautomerizing fused, bicyclic structure is unsaturated and has 5-members or 6-members, provided both rings are not 5-member rings;
X₁ and X₂ are independently selected from O, S, N, NR², CR³, CR⁴═CR⁴¹, CR⁴═N, N═CR⁴, N═N and CR⁴″, provided that (i) when each one of X₁ or X₂ is independently selected from O, S or NR², the other is independently selected from CR³ or N, and (ii) when each one of X₁ or X₂ is independently selected from CR⁴═CR⁴′, CR⁴═N, N═CR⁴ or N═N, the other is independently selected from CR⁴″ or N;
X₃ is independently selected from N, O, S, CR⁵, NR⁵, CR⁵═CR⁵′, CR⁵═N, N═CR⁵ and N═N, and X₄ is independently selected from O, S, N and CH, provided that (i) when each X₃ is independently selected from CR⁵ or N, X₄ is independently selected from O or S, and (ii) when each X₃ is independently selected from O, S, NR⁵, CR⁵═CR⁵′, CR⁵═N, N═CR⁵ or N═N, X₄ is independently selected from CH or N;
T is an amido-containing structure: 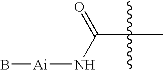
wherein A, when present, is independently selected from —CH₂CH₂C(O)— or —CH₂C(O)—, wherein the terminal methylene group is bound to nitrogen and the terminal carbonyl carbon is bound to B; and, B is independently selected from a diamine or triamine end-group;
Y, when present, is independently selected from H, NH₂, OH, SH, Br, Cl, F, OCH₃, CH₂OH, CH₂SH and CH₂NH₂;
Z is independently selected from (i)—C(O)NH-Q-, wherein Q is independently selected from substituted or unsubstituted C₁₋₆ alkyl, or (ii) one of structures (1), (2), (3) and (4): 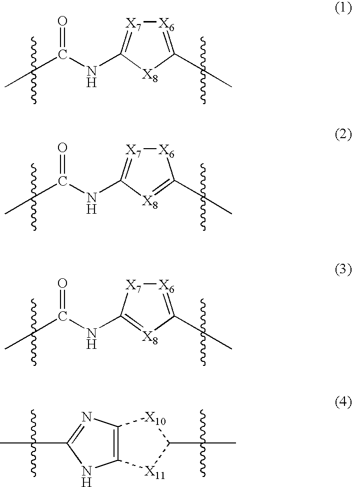
wherein
for structure (1) X₆ is CR⁶, X₇ is independently selected from CR⁷ or N, and X⁸ is independently selected from O or S,
for structure (2) X₆ is independently selected from NR⁶, O or S, X₇ is independently selected from CR⁷ or N, and X⁸ is independently selected from CH, C(OH), or N,
for structure (3) X₆ is independently selected from CR⁶ or N, X₇ is independently selected from NR⁷, O or S, and X⁸ is independently selected from CH, C(OH), or N; and,
for structure (4) each ring is unsaturated, X₁₀ is independently selected from CR¹⁰═CR¹⁰′, CR¹⁰═N, N═CR¹⁰ or N═N, and X₁₁ is independently selected from CH, C(OH), or N;
each substituent R², R³, R⁴, R⁴, R⁴″, R⁵, R⁵′, R⁶, R⁷, R¹⁰ and R¹⁰′ is independently selected from H, hydroxy, N-acetyl, benzyl, substituted or unsubstituted C₁₋₆ alkyl, substituted or unsubstituted C₁₋₆ alkylamine, substituted or unsubstituted C₁₋₆ alkyldiamine, substituted or unsubstituted C₁₋₆ alkylcarboxylate, substituted or unsubstituted C₂₋₆ alkenyl, substituted or unsubstituted C₂₋₆ alkynyl and, when attached to a carbon atom, optionally halo, provided that (i) when X¹ or X² is NR², R² is other than H, and (ii) when X³ is NR⁵, R⁵ is other than H; and,
subscripts a, b, d, e, f, h, i, and p are each, independently, greater than or equal to 0, and subscripts m and q are 0 or 1, provided that (i) when L is not a non-tautomerizing, fused, bicyclic structure, b or f is at least about 1, (ii) when m is 0, q and p are also 0; (iii) the result of [(a+b)*d] is at least about 2; and, (vi) the result of [(e+f)*h] is the same or different from the result of [(a+b)*d] and is greater than or equal to 0, further provided that when the result of [(e+f) *h] is 0, m is 0.

25. The compound of claim 24 wherein L is a non-tautomerizing, fused bicyclic structure: 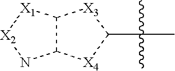
wherein X₁, X₂, X₃ and X₄ are as defined in claim 24.

26. The compound of claim 24 wherein Y is NH₂ and p is 2.

27. The compound of claim 24 wherein Y is OCH₃ and p is 1.

28. The compound of claim 24 wherein L is a non-tautomerizing, fused bicyclic structure: 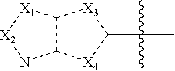
and further wherein X₁, X₂, X₃ and X₄ are as defined in claim 24, and the result of a+b ranges from about 2 to about 8.

29. The compound of claim 24 wherein the result of e+f is the same as the result of a+b.

30. The compound of claim 24 wherein the result of e+f is 0, and further wherein m is 0.

31. The compound of claim 30 wherein T is an amido-containing structure: 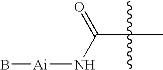
and further wherein B and A are as defined in claim 24, and subscript i is 1.

32. The compound of claim 24 wherein L is a non-tautomerizing, fused bicyclic structure: 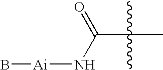
and further wherein X₁ is independently selected from N-methyl, S or O, X₂ is CH, X₃ is CH═CH, and X₄ is CH.

33. The compound of claim 24 wherein b is 1 or more, L is a non-tautomerizing, fused bicyclic structure: 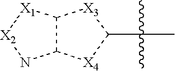
T is an amido-containing structure: 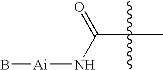
wherein X₁, X₂, X₃, X₄, B, A and subscript i are as defined in claim 24, and a, h and m are each 0, the compound having the formula: 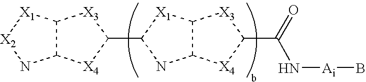
wherein each of X₁, X₂, X₃, and X₄ may be the same or different for each of said fused, bicyclic structures.

34. The compound of claim 24 wherein said compound has the structure: 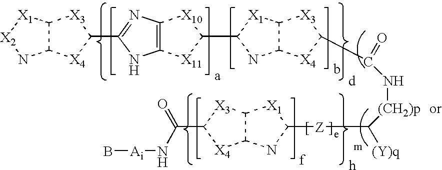
wherein: each of X₁, X₂, X₃, X₄, X₁₀, X₁₁ are as independently defined in claim 24; each of subscripts a, b, d, e, f, h, i, m, p and q are as independently defined in claim 24; and each of Y, A and B are as independently defined in claim 24.

35. The compound of claim 24 wherein said compound has the structure: 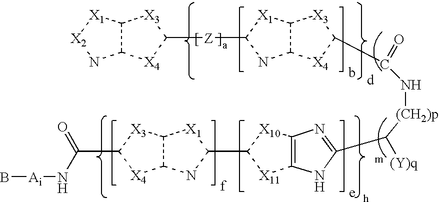
wherein: each of X₁, X₂, X₃, X₄, X₁₀, X₁₁ are as independently defined in claim 24; each of subscripts a, b, d, e, f, h, i, m, p and q are as independently defined in claim 24; and each of Y, A and B are as independently defined in claim 24.

36. The compound of claim 24 wherein the non-tautomerizing, fused, bicyclic structure is: 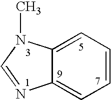
wherein, when (i) said fused bicycle occupies a first terminal position within the compound, carbon C7 forms a bond with the remaining portion of the compound, and (ii) said fused bicyclic structure occupies a non-terminal position within the compound, the heterocyclic ring thereof is the first ring, carbons C₂₋and C7 forming bonds with the remaining portion of the compound.

37. The compound of claim 24 wherein the non-tautomerizing, fused, bicyclic structure is: 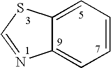
wherein, when (i) said fused bicycle occupies a first terminal position within the compound, carbon C7 forms a bond with the remaining portion of the compound, and (ii) said fused bicyclic structure occupies a non-terminal position within the compound, the heterocyclic ring thereof is the first ring, carbons C2 and C7 forming bonds with the remaining portion of the compound.

38. The compound of claim 24 wherein the non-tautomerizing, fused, bicyclic structure is: 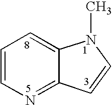
wherein, when (i) said fused bicycle occupies a first terminal position within the compound, carbon C2 forms a bond with the remaining portion of the compound, and (ii) said fused bicyclic structure occupies a non-terminal position within the compound, the heterocyclic ring thereof is the first ring, carbons C2 and C6 forming bonds with the remaining portion of the compound.

39. The compound of claim 24 wherein at least one Z has the structure: 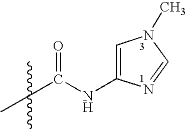
wherein (i) the non-substituted N atom (N1) is directed toward the floor of the minor groove, and (ii) carbon C2 and the carbonyl carbon form bonds with the compound when the moiety occupies an internal position therein.

40. The compound of claim 24 wherein at least one Z has the structure: 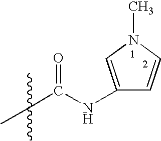
wherein (i) the substituted N atom is directed away from the floor of the minor groove, and (ii) carbon atom C2 and the carbonyl carbon form bonds with the compound when the moiety occupies an internal position therein.

41. The compound of claim 24 wherein the number of bonds separating the H-bond donor atoms from the H-bond acceptor atom is about the same in the compound.

42. The compound of claim 41 wherein the number of bonds is about 5.

43. The compound of claim 1 wherein the heteroatom of the H-bond acceptor moiety of the non-tautomerizing, fused, bicyclic structure is separated from a heteroatom of a H-bond donor moiety by more than two bonds.

44. The compound of claim 43 wherein the heteroatom of the H-bond donor moiety and the heteroatom of the H-bond acceptor moiety are separated by about 5 bonds.

45. The compound of claim 44 wherein substantially all of the H-bond donor moieties and H-bond acceptor moieties in the compound are separated by about 5 bonds from each other.

46. The compound of claim 1 wherein the non-tautomerizing, fused, bicyclic structure has a second heteroatom therein which may optionally act as an H-bond acceptor to bind guanine in the minor groove.

47. The compound of claim 46 wherein said second heteroatom is spaced from the first heteroatom such that, as H-bond interactions between said first heteroatom and a guanine nucleotide decreases, H-bond interactions between said second heteroatom and said guanine nucleotide increases.

48. A synthetic and/or non-naturally occurring polyamide analog for binding a sequence of nucleotides in a minor groove of dsDNA with specificity, said analog comprising at least two synthetic and/or non-naturally occurring compounds as defined by claim 1, which may be the same or different, and which are linked by an aliphatic amino acid moiety which forms a hairpin turn in said polyamide analog.

49. A triplex comprising a sequence of dsDNA which contains at least one guanine nucleotide and to which is bound in a minor groove thereof the synthetic and/or non-naturally occurring polyamide analog as defined by claim 48.

50. A diagnostic kit comprising the synthetic and/or non-naturally occurring polyamide analog of claim 48.

51. A triplex comprising a sequence of dsDNA which contains at least one guanine nucleotide and to which is bound in a minor groove thereof the synthetic and/or non-naturally occurring compound as defined by claim 1.

52. A diagnostic kit comprising the synthetic and/or non-naturally occurring compound of claim 1.

53. A process for preparing a synthetic and/or non-naturally occurring compound on a solid support, said compound comprising at least one H-bond donor moiety and at least one H-bond acceptor moiety which are spaced apart to bind with specificity a nucleotide sequence in a minor groove of dsDNA, wherein said H-bond acceptor moiety has a fused, bicyclic structure and is heteroaromatic, wherein said structure has a heteroatom therein which acts as a hydrogen bond acceptor to bind guanine in the minor groove of the dsDNA sequence, and wherein said structure cannot form a tautomer in which said heteroatom becomes a H-bond donor, the process comprising:
preparing a support for attachment of said compound;
reacting an amino acid with a reagent to provide an amino acid containing an amino group which is protected and a carboxyl group reactive with an amino functionality;
deprotecting the amino acid and adding the protected and reactive amino acids to the solid support beginning with the carboxy terminal amino acid;
cleaving the compound from the resin; and,
purifying the compound;
wherein at least one of said protected and sequentially deprotected amino acids comprises a fused, bicyclic structure having a 5- or 6-member heteroaromatic ring, wherein said structure has a heteroatom therein which acts as a hydrogen bond acceptor to bind guanine in the minor groove of the dsDNA sequence, and further wherein said structure cannot form a tautomer in which said heteroatom becomes a H-bond donor.

54. The process of claim 53 wherein the support is selected from the group consisting of inorganic and polymeric supports.

55. The process of claim 54 wherein the support is an inorganic support selected from the group consisting of silicates, quartz and aluminum.

56. The process of claim 54 wherein the support is polymeric.

57. The process of claim 56 wherein the support is polystyrene.

58. The process of claim 53 wherein the support comprises the surface of a well of a substratum.

59. The process of claim 58 wherein the support comprises the surface of a well of a multi-well substratum.

60. The process of claim 59 wherein the support comprises the surface of a well of a micro-titer plate comprising at least 96 wells.

61. The process of claim 53 wherein said compound further comprises one or more substituted or unsubstituted imidazole groups.

62. The process of claim 61 wherein said compound further comprises one or more substituted or unsubstituted pyrrole groups.

63. The process of claim 53 wherein said amino acid is protected by a t-butoxycarbonyl or 9-fluorenylmethylcarbonyl group.

64. The process of claim 53 wherein said compound comprises one or more N-methyl 4-imidazolecarboxamide or N-methyl-pyrrolecarboxamide moieties.

65. The process of claim 53 wherein said compound is attached to the support though a spacer selected from the group consisting of glycine, β-alanine, glycine-PAM, and glycine-BAM.


SEQUENCE LISTING (1 sequence)

SEQ ID NO: 1 - DNA, 30 bp, Artificial Sequence
  Biotinylated Sequence in Example 8
cgtatgttgtgtgttttcacacaacatacg